(19) United States
(12) Patent Application Publication
(10) Pub. No.: US 2013/0072448 A1
(43) Pub. Date: Mar. 21, 2013

(54) Drug Combination Comprising A Glycolysis Inhibitor And A Tyrosine Kinase Inhibitor

(71) Applicant: URIFER LTD., Raanana (IL)

(72) Inventors: Uri Nir, Moshav Gamzo, D.N. Merkaz (IL); Sally Shpungin, Ramat-Gan (IL)

(73) Assignee: URIFER LTD., Raanana (IL)

(21) Appl. No.: 13/677,231

(22) Filed: Nov. 14, 2012

Related U.S. Application Data
(63) Continuation-in-part of application No. PCT/IL12/50264, filed on Jul. 19, 2012.
(60) Provisional application No. 61/509,259, filed on Jul. 19, 2011.

(51) Int. Cl. A61K 31/7004; A61K 31/496; A61K 31/70

(52) U.S. Cl. CPC A61K 31/7004; A61K 31/70; A61K 31/496
USPC 514/23

(57) ABSTRACT

A drug combination comprising at least one inhibitor of tyrosine kinase and at least one inhibitor of glycolysis. The drug combination may be used in the treatment of proliferative disorders, such as liver or colon cancer. The inhibitor of tyrosine kinase may be an inhibitor of at least one of the tyrosine kinases Fer and FerT.

### TECHNOLOGICAL FIELD

The present invention relates to drug combinations.

### PRIOR ART

References considered to be relevant as background to the presently disclosed subject matter are listed below:
U.S. Pat. No. 7,442,781 to Nir et al.
International Patent Publication WO2007/107991 to Nir et al.
International Patent Publication WO2009/098690 to Nir et al.
International Patent Publication WO2009/019708 to Nir et al.
International Patent Publication WO2010/097798 to Nir et al.
Pasder O, et al. Down regulation of Fer induces PP1 activation and cell-cycle arrest in malignant cells. Oncogene 2006; 25(30):4194-206.
Makovski, A. et al. Down regulation of Fer induces ROS levels accompanied by ATM and p53 activation in colon carcinoma cells Cell. Signal. 2012; 24:1369-1374.
Makovski, A. et al. Intronic promoter drives the BORIS-regulated expression of FerT in colon carcinoma cells. J. Biol. Chem. 2012; 287: 6100-6112.
   Acknowledgement of the above references herein is not to be inferred as meaning that these are in any way relevant to the patentability of the presently disclosed subject matter.

### BACKGROUND

Fer is a tyrosine kinase that resides mainly in the cytoplasm and nucleus of mammalian cells. In the cytoplasm, Fer associates with cell adhesion molecules and its kinase activity increases in growth factor stimulated cells. The function of Fer seems to be redundant in mice, as mice devoid of a functional Fer are viable and fertile. However, Fer has been found to be involved in the proliferation of malignant cell lines. Furthermore, reducing the level of FerT using a specific siRNA in HCT116 colon carcinoma cells lowered both the level of ATP production and the mitochondrial membrane potential in the treated cells.

U.S. Pat. No. 7,442,781 to Nir et al discloses treatment of cancer through detection and modulation of the expression of Fer.

A testis specific variant of Fer, termed FerT, is encoded by an alternatively spliced FER transcript. Fer and FerT share identical SH2 and kinase domains but differ in their N-terminal tails. FerT accumulates in late primary spermatocytes.

International Patent Publication WO2007/107991 of Nir et al discloses methods for modulating a Fer mediated pathway in a cell, for monitoring the effect of a Fer protein in a cell, for altering and inhibiting one or more effects of a Fer protein in a cell.

International Patent Publication WO2009/098690 to Nir et al discloses a Fer-like protein, referred to as “FerC” (Fer colorectal cancer). FerC is a 47 kDa protein having a unique N-terminal sequence and was found to be present in six colon cancer cell-lines and in five hepatocarcinoma (liver cancer) cell-lines, but not in CCD33 normal colon epithelial cells or normal human and mouse fibroblasts. Depletion of FerC impairs cell-cycle progression and induces apoptotic death in treated colon cancer (CC) cells. Recent findings identify FerC with the human testis specific-FerT [Makovski et al. J. Biol. Chem (2012)].

International Patent Publication WO2009/019708 of Nir et al discloses pharmaceutical compositions for use in treating cancer.

International Patent Publication WO2010/097798 of Nir et al discloses heterocyclic compounds for the treatment of disorders, in particular, cancer.

Eukaryotic cells utilize an energy generation system which includes the cytoplasmic anaerobic glycolysis path followed by the aerobic mitochondrial path of oxidative phosphorylation. Cancer cells, however, are known to adopt a re-programmed energy producing system, known as aerobic glycolysis or “the Warburg effect”, in which oxidative phosphorylation in the mitochondria is significantly reduced and the energy supply is shifted towards aerobic glycolysis. However, attempts to attenuate cancer progression by inhibiting glycolysis using such inhibitors of glycolysis as the glucose analog 2-deoxyglucose (a competitive inhibitor of the glycolysis enzyme hexokinase), does not exhibit sufficient efficacy. Thus, cancer cells seem to be able to reactivate the mitochondrial oxidative phosphorylation when the glycolytic energy generation pathway is blocked.

### GENERAL DESCRIPTION

The present invention is based on the novel and unexpected observation that combined exposure of cancer cells to a combination of an inhibitor of tyrosine kinase and an inhibitor of glycolysis suppresses the proliferation and survival of cancer cells at a level that is greater than the suppression observed when cancer cells are exposed to either of these inhibitors alone. Thus, according to one aspect, the invention relates to a drug combination comprising: (a) an effective amount of at least one inhibitor of tyrosine kinase; and (b) an effective amount of at least one inhibitor of glycolysis.

The present invention further provides a pharmaceutical composition comprising an effective amount of at least one inhibitor of glycolysis and an effective amount of at least one inhibitor of tyrosine kinase together with a physiologically acceptable carrier. The pharmaceutical composition may be used in the treatment of cancer, e.g. liver or colon cancer.

In a further aspect, the present invention also provides (i) use of an effective amount of at least one inhibitor of glycolysis for the preparation of a pharmaceutical composition for the treatment of cancer in patients being treated also with an inhibitor of tyrosine kinase; and (ii) use of an effective amount of at least one inhibitor of tyrosine kinase for the preparation of a pharmaceutical composition for the treatment of cancer in patients being treated also with an inhibitor of glycolysis.

Provided by the present invention is also (i) an inhibitor of glycolysis or a composition comprising an inhibitor of glycolysis for use in the treatment of cancer in patients being treated also with an inhibitor of tyrosine kinase, and (ii) an inhibitor of tyrosine kinase or a composition comprising an inhibitor of tyrosine kinase for use in the treatment of cancer in patients being treated also with an inhibitor of glycolysis.

The invention also provides a method for treating, ameliorating, delaying the onset or prophylaxis of cancer comprising administering to patient in need an effective amount of each of two types of inhibitors, one being an inhibitor of tyrosine kinase and the other of glycolysis.

In another aspect, the invention provides a method for the treatment, amelioration, delaying the onset or prophylaxis of a proliferative disorder in a subject being treated with at least one inhibitor of glycolysis. In certain embodiments the method of the invention comprises the step of administering to a subject in need thereof a therapeutically effective amount of at least one inhibitor of tyrosine kinase.

Still further, the invention provides a method for the treatment, amelioration, delaying the onset or prophylaxis of a proliferative disorder in a subject being treated with at least one inhibitor of tyrosine kinase. In some embodiments, the method of the invention comprises the step of administering to a subject in need thereof a therapeutically effective amount of at least one inhibitor of glycolysis.

A further aspect of the invention relates to a kit comprising: (a) at least one inhibitor of tyrosine kinase in a first unit dosage form; and (b) at least one inhibitor of glycolysis in a second unit dosage form.

Any inhibitor of glycolysis may be used in accordance with the invention. The glycolysis inhibitor may be, for example, 2-fluoro-deoxyglucose (GI), or 2-deoxyglucose (DG).

Any inhibitor of tyrosine kinase may be used in accordance with the invention. The tyrosine kinase inhibitor may be, for example, an inhibitor of Fer, an inhibitor of FerT, or an inhibitor of both Fer and FerT. The inhibitor of Fer may be, for example, any one of the Fer inhibitors disclosed in WO2009/019708 or WO 2010/097798 of Nir et al, the content of which is incorporated herein by reference. In particular, the inhibitor of Fer may be a compound referred to herein as “compound 0260”. Compound 0260 is the tartarate salt of 6-(4-isopropyl-phenyl)-2-{4-[(4-methyl-piperazin-1-yl)methyl]piperidin-1-yl}imidazo[2,1-b][1,3,4]thiadiazole, and is referred to as compound “522-0251” in WO 2010/097798.

These and other aspects of the invention will become apparent by the hand of the following drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. shows that down-regulation of Fer or FerT induces reactive oxygen species (ROS) levels in HCT116 CC cells. Fer (white column) or FerT (grey) were knocked down using selective siRNAs and the cellular ROS levels were determined. Non relevant siRNA (siRNA-neg-black) served as a control.

FIG. 2A-2C. show immunoprecipitation analysis of cytoplasmic (cyto) and mitochondrial (Mito) fractions of whole cell extracts (CE2) from Sw620 colon carcinoma cells (FIGS. 2A and 2B) and liver and heart cells (FIG. 2C).

FIG. 3. shows an immuno-staining indicating that Fer resides in the mitochondria of CC cells. HCT116 CC cells were fixed and co-immuno-stained with anti-mitochondria mono-clonal antibody (AE-1 Meridian-green) and with an anti-Fer antibody. Obtained images were processed using the IMRIS application. The Fer fraction co-localized with the mitochondria is indicated with white arrows.

FIG. 4A-4B. show Decreased mitochondrial electron transport chain (ETC) complex I and complex V activity in Fer and FerT depleted malignant cells. Fer or FerT were knocked-down in HCT116 CC cells grown with galactose without glucose, as glycolysis attenuating conditions. Complexes I and V were isolated from the control (siRNA-neg) and siRNA-fer/ferT treated cells. Complex I (FIG. 4A) activity and complex V ATP synthesizing activity (FIG. 4B) were determined using specific Microplate assays (MS141 and MS543—Mito Sciences, respectively).

FIG. 5. shows that 0260 inhibits the auto-phosphorylation of Fer in-vivo. HCT116 CC cells were deprived of serum for 24 h in the presence of 2 μM 0260 or 0.5% DMSO (the solvent). 15 min before harvest cells were treated with 20 mM H₂O₂— for activation and auto-phosphorylation of Fer. Protein lysates were prepared from cells: starved in the presence of DMSO and treated with H₂O₂ (1), starved in the presence of 0260 and treated with H₂O₂ (2), and from untreated cells (3). Fer was immuno-precipitated, resolved in SDS-PAGE and reacted with anti-phosphotyrosine (pY— upper panel) or anti-Fer antibodies (lower panel) in a western blot analysis.

FIG. 6. shows treatment of Hep3B liver cancer cells with 100 μM 2-fluoro-deoxyglucose (GI) and increasing concentrations of compound 0260 for 72 h.

FIG. 7. shows treatment of SW620 colon cancer cells with 100 μM 2-fluoro-deoxyglucose (GI) and increasing concentrations of compound 0260 for 72 h.

FIG. 8. shows the progression of the human liver cancer xenografts in mice following simultaneous administration of an inhibitor of glycolysis (GI) and compound 260.

FIG. 9. shows the progression of the human liver cancer xenografts in mice following simultaneous administration of an inhibitor of glycolysis (DG) and compound 260.

FIG. 10. shows the effect of various combinations of the Fer inhibitor compound 260 and the glycolysis inhibitor DG on normal human foreskin fibroblasts, which served as a non-cancerous control cell population;

FIG. 11. shows the effect of various combinations of the Fer inhibitor compound 260 and the glycolysis inhibitor DG on Hep3B cells.

FIG. 12. shows the effect of various combinations of the Fer inhibitor compound 260 and the glycolysis inhibitor DG on HCT116 cells.

FIG. 13. shows the fraction of dead cells in a population of HCT116 cells under the conditions indicated.

FIG. 14. shows the fraction of dead cells in a population of Hep3B cells under the conditions indicated.

### DETAILED DESCRIPTION OF THE INVENTION

The present invention is based on the novel and unexpected observation that Fer and FerT support the mitochondrial ETC and oxidative phosphorylation (Oxphosph) processes in malignant but not in normal cells. Moreover, the inventors surprisingly showed that combined exposure of cancer cells to a combination of an inhibitor of tyrosine kinase and an inhibitor of glycolysis suppresses the proliferation and survival of cancer cells at a level that is greater than the suppression observed when cancer cells are exposed to either of these inhibitors alone. Furthermore, the suppression in the proliferation and survival of cancer cells by a combination of tyrosine kinase and glycolysis inhibitors is greater than the suppression of proliferation observed in non-cancer cells.

Thus, according to a first aspect, the invention relates to a drug combination comprising: (a) an effective amount of at least one inhibitor of tyrosine kinase; and (b) an effective amount of at least one inhibitor of glycolysis.

The drug combination of the invention provides the use of at least one tyrosine kinase inhibitor in combination with at least one glycolysis inhibitor. The term “in combination with” such as when used in reference to a therapeutic regimen, refers to administration or two or more therapies over the course of a treatment regimen, where the therapies may be administered together or separately, and, where used in reference to drugs, may be administered in the same or different formulations, by the same or different routes, and in the same or different dosage form type.

In certain embodiments, the term “inhibitor of tyrosine kinase” should be understood to encompass one molecule or agent or a combination of two or more molecules or agents with tyrosine kinase inhibitory activity; and the term “inhibitor of glycolysis” should be understood to encompass one molecule or agent or a combination of two or more molecules or agents with glycolysis inhibitory activity.

As noted above, the invention relates to a drug combination comprising a tyrosine kinase inhibitor. Such compound may inhibit the expression or the activity of a tyrosine kinase, specifically, of Fer and FerT activity. The term “inhibition” as referred to herein, relates to the retardation, attenuation, retraining or reduction of a process. More specifically, according to certain embodiments the compositions and also to any of the methods of the invention specifically inhibit the tyrosine kinase activity of Fer and FerT and thereby any signaling pathways mediated by these signaling molecules by any one of about 1% to 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

Similarly, the glycolysis inhibitors used by the invention may lead to the inhibition, retardation, attenuation, retraining or reduction of glycosylation process by any one of about 1% to 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

As indicated above, the drug combination of the invention provides the use of at least one tyrosine kinase inhibitor. The term kinase describes a large family of enzymes that are responsible for catalyzing the transfer of a phosphoryl group from a nucleoside triphosphate donor, such as ATP, to an acceptor molecule. Tyrosine kinases catalyze the phosphorylation of tyrosine residues in proteins. Phosphorylation at tyrosine residues controls a wide range of properties in proteins such as enzyme activity, subcellular localization, and interaction between molecules and therefore mediate variety of signal transduction pathways. Inhibitors of tyrosine kinases as used herein specifically refer to inhibitors of either the expression or the tyrosine kinase activity of these enzymes.

According to certain embodiments, the inhibitor of tyrosine kinase provided by the drug combinations of the invention may be an inhibitor that inhibits the expression or the catalytic activity of at least one of Fer and FerT.

Tyrosine-protein kinase Fer also known as proto-oncogene c-Fer, p94-Fer, Tyrosine kinase 3 (TYK3), acts downstream of cell surface receptors for growth factors and plays a role in the regulation of the actin cytoskeleton, microtubule assembly, lamellipodia formation, cell adhesion, cell migration and chemotaxis. Fer acts downstream of EGFR, KIT, PDGFRA and PDGFRB, promoting activation of NF-kappa-B and cell proliferation.

It should be appreciated that in some embodiments, as used herein in the specification and in the claim section below, Fer protein refers to the human Fer protein. In certain embodiments the human Fer protein is as denoted by the sequence herein referred to as SEQ ID NO. 2 (identifier P16591 RefSeq; NP_{—}0052237.2; GI119964721), encoded by the nucleic acid sequence as denoted by SEQ ID NO. 1 (NM_{—}005246.2; GI119964720)

The isoform of Fer also known as FerT-p4′7, is a truncated form of Fer, produced by alternative promoter usage that leads to deletion of residues 1-412. In certain embodiments the amino acid sequence of the human FerT protein is as denoted by SEQ ID NO. 3 (identifier -hFerT AEY69041; GI 373428613) and encoded by the ferT mRNA denoted by SEQ ID NO. 6 (identifier JQ412173.1; GI 373428612).

Any inhibitor of tyrosine kinase may be used in accordance with the invention. The tyrosine kinase inhibitor may be, for example, an inhibitor of Fer, an inhibitor of FerT, or an inhibitor of both Fer and FerT. The inhibitor of Fer may be, for example, any one of the Fer inhibitors disclosed in WO2009/019708 or WO 2010/097798 of Nir et al, the content of which is incorporated herein by reference.

More specific embodiments for an inhibitor that inhibits the activity of at least one of Fer and FerT is a compound of formula (I) or pharmaceutically acceptable salts thereof:

Thus, for example, the kinase inhibitor may be a compound of formula (I) or pharmaceutically acceptable salts thereof:

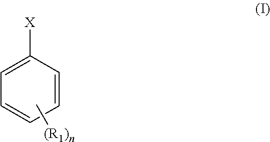

wherein X is selected from the following:

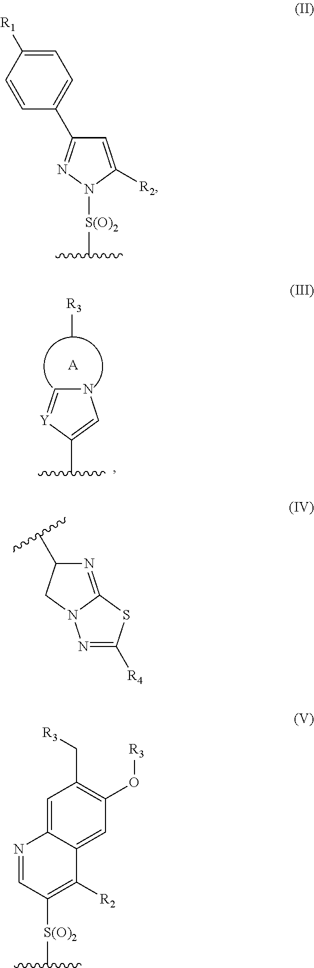

wherein R₁ is independently selected from H, F, Cl, Br, I or a C₁₋₅ linear or branched alkyl; n is 1, 2, 3, 4 or 5; R₂ is NI₂, R being independently hydrogen or linear or branched C₁₋₅alkyl group; R₃ is a linear or branched C₁₋₅alkyl group; R₄ is a group of formula

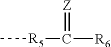

R₅ being a 5- or 6-membered aromatic or non-aromatic ring optionally having one, two or three heteroatoms selected from O, N or S; R₆ being a 5- or 6-membered aromatic or non-aromatic ring optionally having one, two or three heteroatoms selected from O, N or S optionally having one or two substituents independently selected from halogen and a linear or branched C₁₋₅alkyl group; Z is selected from O or S; Y is C—H or N; and A is a 5- or 6-membered fused aromatic or non aromatic ring optionally being a heterocyclic ring comprising 1 to 3 heteroatoms selected from O, N or S;
C₁₋₅ Linear or branched alkyl means a methyl, ethyl, propyl, isopropyl, n-butyl, isobutyl, sec.-butyl, tert.-butyl, n-pentane, iso-pentane, sec.-pentane or tert.-pentane that may optionally be partially substituted by halogen selected from F, Cl, Br, or I.

As a specific example, the tyrosine kinase inhibitor may be a compound, having chemical formula C₁₅H₇ClF₅N₃O₂S and (VI):

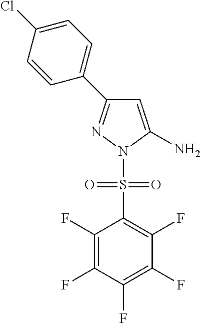 [SMILES: Nc1cc(-c2ccc(Cl)cc2)nn1S(=O)(=O)c1c(F)c(F)c(F)c(F)c1F]

As another example, the tyrosine kinase inhibitor may be a compound having the chemical formula C₁₃H₁₂FN₃S and (VII):

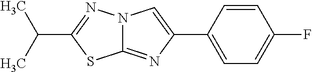 [SMILES: CC(C)c1nn2cc(-c3ccc(F)cc3)nc2s1]

As yet another example, the kinase inhibitor may be a compound having the chemical formula C₁₅H₁₂FN and (XIII):

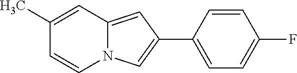 [SMILES: Cc1ccn2cc(-c3ccc(F)cc3)cc2c1]

In yet another preferred embodiment, the pharmaceutical composition of the invention comprises a compound having the chemical formula C₂₁H₂₄N₂O₄S and (IX):

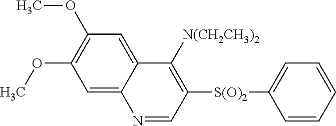

In still another example, the tyrosine kinase inhibitor may be a compound having the chemical formula C₂₂H₂₈N₆OS (X):

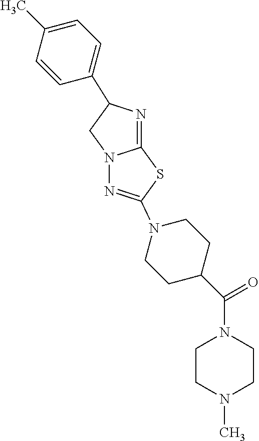 [SMILES: Cc1ccc(C2CN3N=C(N4CCC(C(=O)N5CCN(C)CC5)CC4)SC3=N2)cc1]

As still further examples, the tyrosine kinase inhibitor may be a compound of formulae (XI) or (XII) or pharmaceutical acceptable salts thereof:

wherein R₁, R₂ and R₃ are independently selected from hydrogen, halogen, C₁₋₆alkyl, C₂₋₆alkenyl, N—C₁₋₆alkyl, N—C₂₋₆alkenyl the C₁₋₆alkyl and C₂₋₆alkenyl being straight or branched.

The tyrosine kinase may be a compound of formula (XI) or (XII) or pharmaceutical acceptable salts thereof:

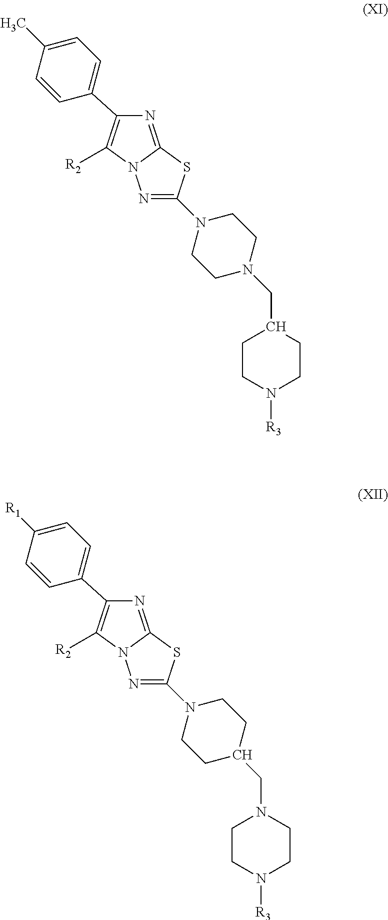

wherein R₁ is hydrogen, methyl, ethyl, propyl, isopropyl, N-isopropyl, butyl, sec.-butyl, tert.-butyl, N-butyl, N-sec.butyl, N-trt.-butyl, F, Cl, Br, I; R₂ is hydrogen, methyl, ethyl, propyl, isopropyl, butyl; R₃ is hydrogen, methyl, ethyl, propyl, isopropyl, butyl.

As a specific example, the tyrosine kinase inhibitor may be a compound of formula (XI) or (XII) wherein R1 is isopropyl, R3 is CH3, and R2 is H. The tyrosine kinase of formula (XII) wherein R1 is isopropyl, R3 is CH3, and R2 is H is referred to herein as “compound 260”. Compound 0260 is the tartarate salt of 6-(4-isopropyl-phenyl)-2-{4-[(4-methyl-piperazin-1-yl)methyl]piperidin-1-yl}imidazo[2,1-b][1,3,4]thiadiazole, and is referred to as compound “522-0251” in WO 2010/097798.

Acceptable pharmaceutical salts are selected from suitable pharmaceutically acceptable salts of the compounds of the invention include acid addition salts formed with pharmaceutically acceptable organic or inorganic acids, for example hydrochlorides, hydrobromides, sulphates, alkyl- or arylsulphonates (e.g. methanesulphonates or p-toluenesulphonates), phosphates, acetates, citrates, succinates, tartrates, trifluoroacetates, lactates, fumarates, malates and maleates.

The pharmaceutical compositions according to the present invention may be in a solid form of capsules, tablets; may be in a form suitable for topical administration as ointments, creams, lotions, gels. Alternatively, it may be in the form of drops, syrups, suspensions, injectable powders or liquid in ampoules.

When present in a liquid form, the active component according to the present invention is dissolved in a solvent or mixture of solvents that are allowed for in vivo use (GRAS). In particular, DMSO, Cyclodextrins, such as α-Cyclodextrin, 2-hydroxypropyl-β-cyclodextrin, 3-hydroxypropyl-β-cyclodextrin, 4-Sulfo-butyl-cyclodextrin solutol, cremophor were used. The compositions may comprise carbohydrates such as, lactose, dextrose, sucrose, trehalose, dextrates.

It should be appreciated that the invention further encompasses the use of tyrosine kinase inhibitors that inhibit, or down regulate the expression of at least one tyrosine kinase. In more specific embodiments, the invention encompasses the use of an inhibitor that down regulates the expression of at least one of Fer and FerT.

The term “down-regulation” or “down-regulates” as used herein refers to causing, directly or indirectly, restriction, retardation, reduction, decrease or diminishing the expression of a desired gene product encoding a target protein. Expression refers to the production of a gene product in an organism or a cell. More specifically, down regulation relates to reduction in the transcription of a desired gene, specifically, the genes encoding any one of Fer and FerT. Reduction in the amount, stability or translatability of transcription products (e.g. RNA) of the gene, and/or reduction in translation of the polypeptide(s) encoded by the desired gene, e.g., at least one of Fer and FerT.

In some embodiments, the inhibitor used by the invention may be an agent that down regulates the expression of at least one of Fer and FerT by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding target gene, specifically, the genes encoding Fer and FerT. As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

More specifically, the dsRNA encompassed by the invention may be selected from the group consisting of small interfering RNA (siRNA), MicroRNA (miRNA) and short hairpin RNA (shRNA). As known in the art and as detailed herein above, RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs containing a sequence complementary to one of the siRNA strands, thereby down regulating the expression of a specific target gene.

As exemplified by Example 1, the inventors used siRNA molecules that specifically eliminate Fer and FetT expression. Thus, in certain specific embodiments, such inhibitors may be siRNA molecules. In some specific and non-limiting embodiments, such siRNA may be a siRNA specific for Fer, as denoted by SEQ ID NO. 4. In yet another embodiment, the invention provides the use of an siRNA molecule specific for FerT, as denoted by SEQ ID NO. 5.

The invention further encompasses the use of other agents that specifically down regulate the expression of any one of Fer and FetT, for example, double-stranded RNA (dsRNA), an antisense RNA, a single-stranded RNA (ssRNA), triplex forming oligonucleotides (TFOs) a Ribozyme and DNAzyme.

It must be appreciated that all the inhibitors described herein above for the drug combinations of the invention are also applicable for any aspect of the invention, specifically for the uses, compositions, methods, dosage unit forms and kits of the invention.

As indicated above, the drug combination of the invention further comprises an inhibitor of glycolysis.

Glycolysis as used herein is the metabolic pathway that converts glucose C6H12O6, into pyruvate, CH3COCOO—+ H+. The free energy released in this process is used to form the high-energy compounds ATP (adenosine triphosphate) and NADH (reduced nicotinamide adenine dinucleotide). Glycolysis takes place in the cytoplasm and requires anaerobic conditions, while the cellular respiration that takes place in the mitochondria. Glycolysis occurs in nearly all organisms, both aerobic and anaerobic. Other monosaccharides, such as fructose and galactose, can also be substrates in glycolysis. An intermediate in glycolytic pathway, dihydroxyacetone phosphate (DHAP), is a source of the glycerol that combines with fatty acids to form fat.

Enhanced glycolysis represents a striking feature of cancers and can therefore serve to indicate a malignant transformation. As indicated in the background of the invention, the Warburg effect is the observation that most cancer cells predominantly produce energy by a high rate of glycolysis followed by lactic acid fermentation in the cytosol, rather than by a comparatively low rate of glycolysis followed by oxidation of pyruvate in mitochondria as in most normal cells. The latter process is aerobic. Malignant, rapidly growing tumor cells typically have glycolytic rates up to 200 times higher than those of their normal tissues of origin. As indicated herein before, any inhibitor of glycolysis may be used in accordance with the invention.

Glycolysis inhibitors as referred to herein relates to different down-regulators of glycolysis in vitro and in vivo, including SB-204990, 2-deoxy-D-glucose (2DG), 3-bromopyruvate (3-BrPA, bromopyruvic acid, or bromopyruvate), 3-BrOP, 5-thioglucose and dichloroacetic acid (DCA). Such inhibitors are currently the subject of intense research as anticancer agents and clinical trials are ongoing for 2-DG and DCA.

Such glycolysis inhibitor may be in certain embodiments at least one of 2-fluoro-deoxyglucos (GI) and 2-deoxyglucose (DG).

In one embodiment, the glycolysis inhibitor used by the invention may be DG. DG (2-deoxyglucose) (DG) is a glucose molecule which has the 2-hydroxyl group replaced by hydrogen, so that it cannot undergo further glycolysis. DG acts to inhibit the phosphorylation of glucose to produce glucose-6-phosphate in the glycolysis cycle, therefore inhibiting the production of ATP.

In yet another embodiment, the glycolysis inhibitor used by the invention may be GI. GI (2-Fluoro-deoxyglucose) is commonly abbreviated to FDG, is glucose analog with the positron-emitting radioactive isotope fluorine-18 substituted for the normal hydroxyl group at the 2′ position in the glucose molecule. GI is transported intracellularly and phosphorylated by hexokinase to FDG-6-PO4 through the same cellular membrane transport pathways as glucose. Unlike glucose, FDG-6-PO4 is subsequently trapped intracellularly due to lack of further metabolism from insufficient amounts of glucose phosphatase.

GI and DG have been used for cancer imaging, prognosis and evaluation of therapies and antimetabolite activity respectively in a variety of cancers.

It should be appreciated that the invention further encompasses any combination of glycolysis inhibitors described herein.

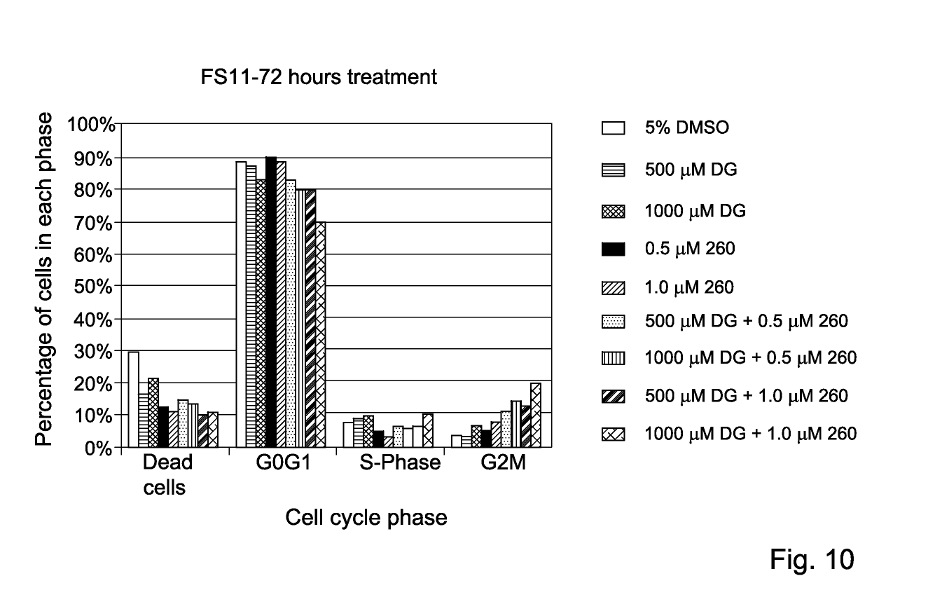

As exemplified in FIGS. 10-14, the combined use of glycolysis inhibitors and inhibitors of tyrosine kinase, resulted in a clear increase in specific death of cancerous cells, probably due to increased apoptosis. Therefore, use of the different drug combinations as well as the combined compositions and unit dosage forms provided by the invention may be employed for modulating cellular apoptosis rates of cancer cells and thereby as an effective treatment of proliferative diseases. Thus, in other embodiments, the drug combinations as well as the compositions and methods of the invention may also lead to an increase, induction or elevation in apoptosis of treated cancer cells, said increase, induction or elevation of apoptosis may be an increase of about 5%, 10%, 15%, 20%, 25%, 30%, 35%, 40%, 45%, 50%, 55%, 60%, 65%, 70%, 75%, 80%, 85%, 90%, 95% or 99% as compared to untreated control.

As mentioned above, specific increase in the apoptosis of cancer cells may be employed for the treatment of proliferative disorders. Thus, according to some embodiments, the drug combination of the invention may be used for the treatment, amelioration, delaying the onset or prophylaxis of a proliferative disorder.

As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. It should be appreciated that treating proliferative disorders as used herein is also meant to embrace any disorder or condition associated with the specific treated proliferative condition.

It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

Malignancy, as contemplated in the present invention may relate to any one of carcinomas, melanomas, lymphomas, leukemias, myeloma and sarcomas.

Carcinoma as used herein, and will be described in more detail herein after in connection with colorectal carcinoma (CRC) and breast cancer, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors including GI tract, colon, lung, liver, breast, prostate and pancreas. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

As demonstrated by Example 3, the drug combination of the invention is particularly applicable for treating liver carcinoma and colon carcinoma.

Thus, according to some embodiments, the drug combination of the invention may be used for treating colon cancer. Colon cancer (also referred to herein as “colorectal cancer or carcinoma”) as herein defined is a disease in which malignant (cancer) cells form in the tissues of the colon. Colorectal carcinoma is the third most common cancer in the United States after prostate and lung/bronchus cancers in men and after breast and lung/bronchus cancers in women.

Risk factors of colon cancer include age and health history, a family history of colon cancer, and in some cases having inflammatory bowel disease (IBD).

The prognosis of patients with colon cancer is clearly related to the degree of penetration of the tumor through the bowel wall, the presence or absence of nodal involvement, and the presence or absence of distant metastases, with these three characteristics forming the basis for all staging systems developed for this disease.

Thus, the drug combination, as well as the combined compositions, methods and unit dosage forms of the invention may be used for the treatment of any type of colorectal cancer (CRC). More specifically, collectively, colon cancer is defined as a cancer that forms in the tissues of the colon (the longest part of the large intestine). These tumors are sometimes referred to as “colorectal” cancer, reflecting the fact that the rectum, the end portion of the colon, may also be affected.

More than 90% of colorectal carcinomas are adenocarcinomas, originating from epithelial cells of the colorectal mucosa. Other rare types of colorectal carcinomas include neuroendocrine, squamous cell, adenosquamous, spindle cell and undifferentiated carcinomas. Conventional adenocarcinoma is characterized by glandular formation, which is the basis for histologic tumor grading.

In yet further embodiments, the drug combination of the invention may be used for treating hepatocarcinoma. Hepatocarcinoma (also Hepatocellular carcinoma (HCC) or malignant hepatoma) as used herein, is the most common type of liver cancer. In many cases, HCC is secondary to viral hepatitis infection (hepatitis B or C); cirrhosis (alcoholism being the most common cause of hepatic cirrhosis); aflatoxin exposures (naturally occurring mycotoxins that are produced by many species of Aspergillus); type 2 diabetes (higher circulating insulin concentration in diabetics with poor insulin control); hemochromatosis (accumulation of iron in the body, primarily due to transfusional iron overload resulting from repeated blood transfusions or hereditary haemochromatosis, a genetic disorder); and Wilson's disease (an autosomal recessive genetic disorder associated with mutations in the ATP7B gene in which copper accumulates in tissues).

HCC symptoms include jaundice, bloating from ascites (peritoneal cavity fluid accumulation), easy bruising from blood clotting abnormalities or as loss of appetite, unintentional weight loss, abdominal pain, especially in the upper-right part, nausea, emesis, or fatigue. HCC can have three distinct patterns of growth: a single large tumor, multiple tumors or poorly defined tumor with an infiltrative growth pattern. HCC treatment and prognosis are dependent on many factors but especially on tumor size and staging.

The invention further provides drug combinations, combined compositions and methods for treating any proliferative disorder that is also affected by enhanced glycolysis. In yet another specific embodiment, the invention may be applicable for treating prostate cancer. Prostate cancer (PC) as used herein is a type adenocarcinoma or glandular cancer developing in the prostate. In most cases, PC develops after the age of 50 and is slow growing, however, there are aggressive PC in which the cancer cells may metastasize, particularly to the bones and lymph nodes. In addition, a condition known as prostatic carcinoma in situ or prostatic intraepithelial neoplasia (PIN) has been closely associated with PC. The drug combination of the invention may be also applied for treating said disorders.

Still further, the drug combinations, as well as the combined compositions and methods of the invention may be used for treating breast cancer. Breast cancer (BC) originates most commonly from the inner lining of milk ducts (ductal carcinoma) or the lobules (lobular carcinoma). While the overwhelming majority of BC occurs in women, male-BC can also occur. Certain breast changes, such as atypical hyperplasia and lobular carcinoma in situ found in benign breast conditions such as fibrocystic breast changes are correlated with an increased risk to BC. BC symptom complex also includes inflammatory BC, Paget's disease of the breast and a rare phyllodes tumor of the breast.

BC may be classified by several grading systems, which influence the prognosis and can affect treatment response. Description of BC optimally includes: histopathology (as ductal or lobular carcinoma, carcinoma in situ or invasive carcinoma); grading (as differentiated breast cells—low grade, moderately differentiated—intermediate grade, or poorly differentiated—high grade), staging (according to TNM system using tumor size, tumor has spread to the lymph nodes and the presence of metastases, common sites include bone, liver, lung and brain). Stage 0 is a pre-cancerous or marker condition, either ductal carcinoma in situ (DCIS) or lobular carcinoma in situ (LCIS); Stages 1-3 are within the breast or regional lymph nodes; Stage 4 is ‘metastatic’ cancer with poor prognosis. Another feature of BC type is a receptor status (estrogen receptor (ER), progesterone receptor (PR) and HER2+/−). ER+ cancers can be treated with estrogen blocking drugs (e.g. tamoxifen). HER2+ responds to the monoclonal antibody trastuzumab (in combination with conventional chemotherapy). Triple negative BC may express androgen receptor and prolactin receptor. It should be appreciated that the invention may be useful in treating any of the breast cancer disorders and stages described herein above.

As indicated above, using a drug combination described above, the invention provides the combined exposure of a treated subject to both inhibitors. The terms “combined exposure” or “combination” used herein refer to a combined treatment of the two types of inhibitors—inhibitor of tyrosine kinase and an inhibitor of glycolysis—in cancer patients. Such a combined treatment may mean that the two types of inhibitors are administered substantially together (formulated together into one composition are each formulated separately and administered to patients at substantially the same time or one followed shortly after the other). Such combined treatment may also include administration of one type of inhibitor at one therapeutic regimen and the other at another therapeutic regimen over the same time period; for example, one type of inhibitor is given to patient daily and another weekly over the course of several weeks or months. In essence, the combined treatment mean that the therapeutic effect is a result of the action of both types of inhibitors, each typically administered at its optimal or accepted therapeutic regimen that may be determined in clinical studies.

Therefore, in a second aspect, the invention provides the use of a therapeutically effective amount of at least one tyrosine kinase inhibitor for the preparation of a composition for the treatment of a proliferative disorder in a subject being treated with at least one glycolysis inhibitor.

Still further, the invention provides the use of a therapeutically effective amount of at least one glycolysis inhibitor for the preparation of a composition for the treatment of a proliferative disorder in a subject being treated with at least one tyrosine kinase inhibitor.

In a further aspect, the invention relate to at least one inhibitor of tyrosine kinase or a composition comprising the same for use in the treatment of a proliferative disorder in a subject being treated with at least one glycolysis inhibitor.

The invention further provides at least one inhibitor of glycolysis or a composition comprising the same for use in the treatment of a proliferative disorder in a subject being treated with at least one tyrosine kinase inhibitor.

According to certain embodiments, at least one inhibitor of tyrosine kinase used by the invention may be an inhibitor of at least one of Fer and FerT. It should be appreciated that any of the inhibitors described for the drug combinations of the invention may be also employed in any use according to the invention.

According to certain embodiments, the tyrosine kinase inhibitor used by the invention is a compound of formula (II) or (III) wherein R1 is isopropyl, R3 is CH3, and R2 is H.

Still further, the inhibitor of glycolysis used by the invention may be at least one of 2-fluoro-deoxyglucos (GI) and 2-deoxyglucose (DG).

Another aspect of the invention relates to a pharmaceutical composition comprising an effective amount of at least one inhibitor of tyrosine kinase, an effective amount of at least one inhibitor of glycolysis and a physiologically acceptable carrier.

In certain embodiments the tyrosine kinase inhibitor comprised within the composition of the invention may be a compound that inhibits the catalytic activity of Fer and FerT. In more specific embodiments such compound may be a compound of formula (XI) or (XII) wherein R1 is isopropyl, R3 is CH3, and R2 is H. According to these embodiments, an inhibitor of glycolysis may be at least one of 2-fluoro-deoxyglucos (GI) and 2-deoxyglucose (DG).

According to some embodiments, the composition of the invention may be particularly useful in the treatment, amelioration, delaying the onset or prophylaxis of a proliferative disorder. It should be appreciated that the composition of the invention may be applicable for any proliferative disorder described by the invention.

In more specific embodiments, the composition of the invention may be used for treating a proliferative disorder that may be any one of liver carcinoma and colon carcinoma.

Still further, the compositions of the invention comprise any effective amount of both compounds. Such effective amount may be adapted to provide 0.1-100 mg/Kg of body weight of the tyrosine kinase inhibitors and about 0.1-100 mg/Kg of body weight the glycolysis inhibitors used by the invention. Specifically, in embodiments where the compound 0260 is used as the tyrosine kinase inhibitor, either 0.1-50 mg/Kg, 0.2-40 mg/Kg, 0.3-30 mg/Kg, 0.4-20 mg/Kg 0.5-100 mg/Kg or 0.5-50 mg/Kg may be used. More specifically, said effective amount may range from between about 0.4 to about 4 mg/Kg of compound 0260. According to other embodiments, the amount of compound 0260 may range between about 0.5 to 50 mg/Kg, and more specifically, about 0.5, 1, 1.5, 2, 2.5, 3, 3.5, 4, 4.5, 5, 5.5, 6, 6.5, 7, 7.5, 8, 8.5, 9, 9.5, 10, 20, 30, 40, 50 mg/Kg and even more.

In certain embodiments, where a combined composition uses 0260 in combination with GI as an inhibitor of glycolysis, the effective amount of compound 0260 may range from about 0.1 to about 10 mg/Kg, about 0.2 to about 8 mg/Kg, about 0.3 to about 6 mg/Kg, about 0.4 to about 4 mg/Kg, specifically, about 1, 0.9, 0.8, 0.7, 0.6, 0.5, 0.4, 0.3, 0.2, 0.1, and more specifically, about 0.4 mg/Kg. According to other embodiment, the amount of compound 0260, where used in combination with GI, may range between about 0.5 to about 10 mg/Kg, and specifically, about 0.5, 1, 1.5, 2, 2.5, 3, 3.5, 4, 4.5, 5, 5.5, 6, 6.5, 7, 7.5, 8, 8.5, 9, 9.5, 10 and more specifically, about 5 mg/kg and more. In other embodiments, where the combined treatment includes DG, the effective amount used for compound 0260 may range between about 0.1 to about 100 mg/Kg, about 0.9 to about 30 mg/Kg, about 1 to about 20 mg/Kg, specifically, about 1 to about 10 mg/Kg, specifically, about 10, 9, 8, 7, 6, 5, 4, 3, 2, 1 and more specifically, about 4 mg/Kg. According to other embodiment, the amount of compound 0260, where used in combination with DG may range between about 1 to about 100 mg/kg, specifically, 50 mg/kg.

In some embodiments, an effective amount of GI in the combined composition of the invention may range between about 0.1 to about 20 mg/Kg, about 0.2 to about 8 mg/Kg, about 0.3 to about 6 mg/Kg, about 0.4 to about 4 mg/Kg, about 0.5 to about 2 mg/Kg, about 0.6 to about 1.5 mg/Kg, and specifically, about 1, 1.1, 1.2, 1.3, 1.4, 1.5, 1.6, 1.7, 1.8, 1.9, 2, and more specifically, about 1.2 mg/Kg. In an alternative embodiment, the amount of GI may range between about 1 to about 20, specifically, about 1, 2, 3, 4, 5, 6, 7, 8, 9, 10, 11, 12, 13, 14, 15, 16, 17, 18, 19, 20 mg/kg and even more. In more specific embodiment, the amount of GI may be around 15 mg/kg/ In other embodiments, an effective amount of DG in the compositions of the invention may range between about 0.1 to about 500 mg/Kg, about 0.2 to about 40 mg/Kg, about 0.3 to about 30 mg/Kg, about 0.4 to about 20 mg/Kg, about 0.5 to about 18 mg/Kg and specifically, about 20, 19, 18, 17, 16, 15, 14, 13, 12, 11, 10 and more specifically, about 16 mg/Kg. In yet other alternative embodiments, the amount of DG may range between about 10 to 500 mg/kg, specifically, about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 and 500 mg/kg. In more specific embodiments, the amount of DG may be around 200 mg/kg.

It must be noted that the above effective amount relate to an amount given in each administration. As shown by the Examples, these effective amounts of active compounds were administered twice a day. Therefore, in certain embodiments, the daily effective amount of compound 0260 when used in combination with GI as an inhibitor of glycolysis, may be about 0.8 mg/Kg, and when used in combination with DG, the amount may be about 8 mg/Kg. As to GI, in certain specific embodiments, the daily amount may be about 2.4 mg/Kg, and for DG, the daily effective amount may be about 32 mg/Kg. Nevertheless, it should be further appreciated that the ultimate dose comprised within the composition of the invention will of course depend on the condition being treated, the route of administration and the age, weight and condition of the patient and will be at the doctor's discretion.

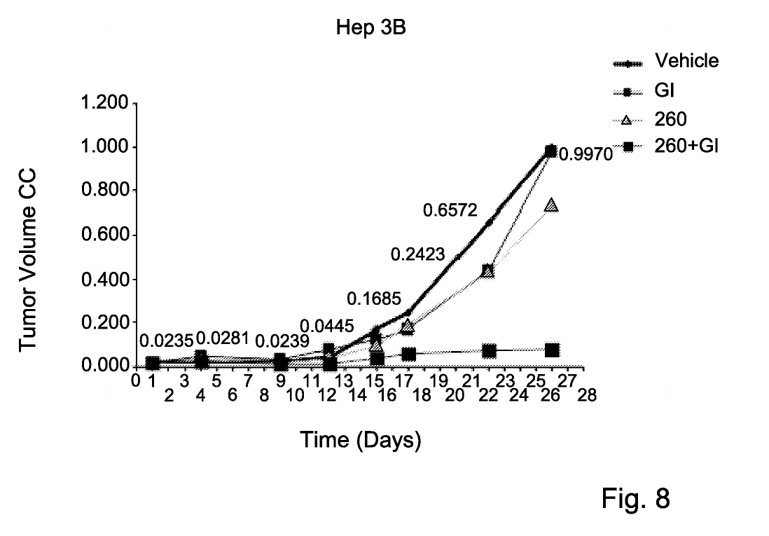

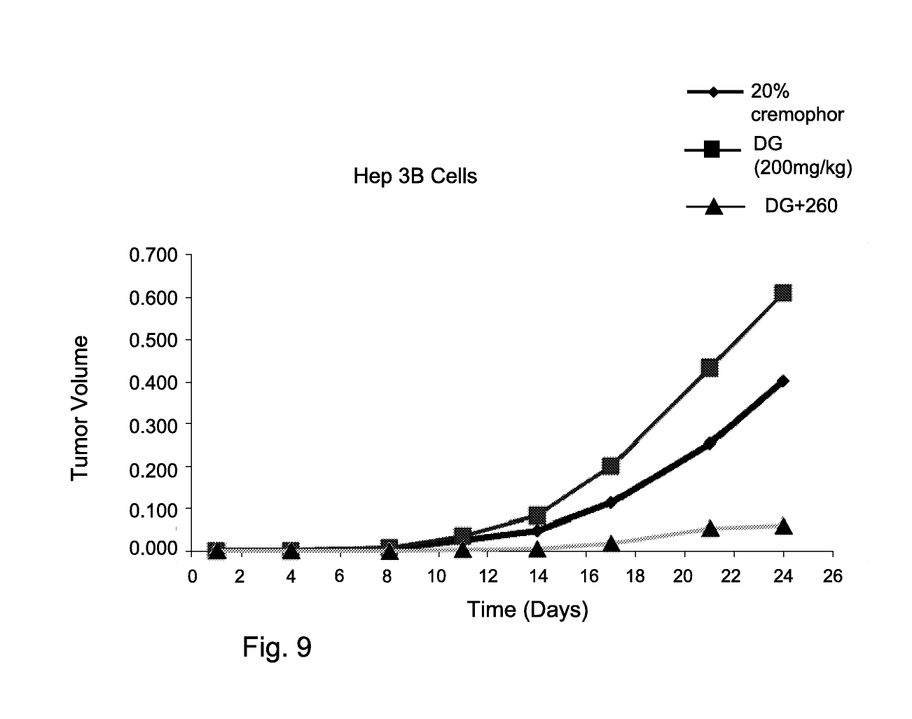

It should be appreciated that the dosage of the active compounds used by the invention using the mouse models as demonstrated by FIGS. 8 and 9, where converted to the appropriate doses for treating a human subject. Such doses can be provided in a single dose or as a number of discrete doses.

All compositions of the invention described herein and herein after, may comprise pharmaceutically acceptable carrier or excipient. As used herein “pharmaceutically acceptable carrier or excipient” includes any and all solvents, dispersion media, coatings and antifungal agents and the like. The use of such media and agents for pharmaceutical active substances is well known in the art. Except any conventional media or agent incompatible with the active ingredient, its use in the therapeutic composition is contemplated.

Pharmaceutically acceptable salts, for example, refer to the non-toxic alkali metal, alkaline earth metal, and ammonium salts commonly used in the pharmaceutical industry including the sodium, potassium, lithium, calcium, magnesium, barium, ammonium, and protamine zinc salts, which are prepared by methods well known in the art. The term also includes non-toxic acid addition salts, which are generally prepared by reacting the compounds of this invention with a suitable organic or inorganic acid. Representative salts include the hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, napsylate, trifluoroacetate and the like.

Pharmaceutically acceptable acid addition salt are those salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as trifluoroacetic acid, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, menthanesulfonic acid, ethanesulfonic acid, p-toluenesulfonic acid, salicylic acid and the like.

Pharmaceutically acceptable esters are those esters which retain, upon hydrolysis of the ester bond, the biological effectiveness and properties of the carboxylic acid or alcohol and are not biologically or otherwise undesirable. These esters are typically formed from the corresponding carboxylic acid and an alcohol. Generally, ester formation can be accomplished via conventional synthetic techniques.

Pharmaceutically acceptable amides are those amides which retain, upon hydrolysis of the amide bond, the biological effectiveness and properties of the carboxylic acid or amine and are not biologically or otherwise undesirable. These amides are typically formed from the corresponding carboxylic acid and an amine. Generally, amide formation can be accomplished via conventional synthetic techniques.

“Pharmaceutically or therapeutically acceptable carrier” refers to a carrier medium which does not interfere with the effectiveness of the biological activity of the active ingredients and which is not toxic to the host or patient.

In some particular embodiments, the composition of the invention is specifically suitable for intraperitoneal administration, however it should be noted that the administration may include oral, parenteral, intravenous, intramuscular, subcutaneous, transdermal, intranasal, mucosal, topical or subcutaneous administration, or any combination thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art. Methods of preparing various pharmaceutical compositions with a certain amount of active ingredient are known, or will be apparent in light of this disclosure, to those skilled in this art.

It should be noted that the drug combinations, combined compositions or any unit dosage forms provided by the invention may be also administered orally. The combined compounds employed in the instant therapy can be administered in various oral forms including, but not limited to, tablets, capsules, pills, powders, granules, elixirs, tinctures, suspensions, syrups, and emulsions. It is contemplated that drug combinations of the invention can be delivered by any pharmaceutically acceptable route and in any pharmaceutically acceptable dosage form. These include, but are not limited to the use of oral conventional rapid-release, time controlled-release, and delayed-release pharmaceutical dosage forms. The drug combinations of the invention can be administered in a mixture with suitable pharmaceutical diluents, excipients or carriers (collectively referred to herein as “carrier” materials) suitably selected to with respect to the intended form of administration. As indicated, it is contemplated that oral administration can be effectively employed. Thus, tablets, capsules, syrups, and the like as well as other modalities consistent with conventional pharmaceutical practices can be employed.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

As indicated above, in addition to the intraperitoneal route, the drug combinations and combined compositions used in the uses, methods and kits of the invention may be adapted for administration by any other appropriate route, for example by the parenteral, oral (including buccal or sublingual), rectal, topical (including buccal or sublingual) or vaginal route. Such formulations may be prepared by any method known in the art of pharmacy, for example by bringing into association the active ingredient with the carrier(s) or excipient(s).

Pharmaceutical formulations adapted for rectal administration may be presented as suppositories or enemas.

Pharmaceutical formulations adapted for vaginal administration may be presented as pessaries, tampons, creams, gels, pastes, foams or spray formulations.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood, that the formulations and compositions described herein above, include any combined composition of the invention comprising both, at least one tyrosine kinase inhibitor and at least one glycolysis inhibitor, and for any compositions comprising either the tyrosine kinase inhibitors or the glycolysis inhibitors according to the invention.

Another aspect of the invention relates to a method for the treatment, amelioration, delaying the onset or prophylaxis of a proliferative disorder. In certain embodiments, the method of the invention comprises the step of administering to a subject in need thereof a therapeutically effective amount of at least one inhibitor of tyrosine kinase and an effective amount of at least one inhibitor of glycolysis, any combination thereof or any composition comprising the same.

The term “treatment or prevention” as used herein refers to the complete range of therapeutically positive effects of administrating to a subject including inhibition, reduction of, alleviation of, and relief from, proliferative disorder and illness, cancer symptoms or undesired side effects proliferative disorders. More specifically, treatment or prevention includes the prevention or postponement of development of the disease, prevention or postponement of development of symptoms and/or a reduction in the severity of such symptoms that will or are expected to develop. These further include ameliorating existing symptoms, preventing additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

More specifically, as indicated above, the invention described herein encompasses methods for the treatment of subjects in need thereof. The term “treatment” concerns improvement of at least one undesired manifestation of the disease such as: increase in disease free periods, decrease in acute disease periods (in time and severely), decrease in severity of the disease, improvement in life quality, decreased mortality, decrease in the rate of disease progression as well as prophylactic treatment before disease occurs.

The term “preventing” refers to preventing the occurrence or reoccurrence of the acute disease symptoms. It should be emphasized that the method of the invention is therefore also prophylactic, especially for the treatment of cancer, and includes the administration of the drug combination, combined compositions, unit dosage forms of the invention in order to prevent cancer symptoms and metastasis either on a regular basis or according to need.

The present invention relates to the treatment of subjects, or patients, in need thereof. By “patient” or “subject in need” it is meant any organism who may be affected by the above-mentioned conditions, and to whom the treatment and diagnosis methods herein described is desired, including humans, domestic and non-domestic mammals such as canine and feline subjects, bovine, simian, equine and murine subjects, rodents, domestic birds, aquaculture, fish and exotic aquarium fish, any reptile or zoo animal. More specifically, the drug combinations, composition, combined compositions and methods of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. It should be further noted that particularly in case of human subject, administering of the drug combination to the patient includes both self-administration and administration to the patient by another person.

As noted above, the method of the invention may be specifically used for treating proliferative disorders. It must be understood that all the disorders disclosed by the invention herein before, are also applicable for the methods of the invention. In some embodiments, the method of the invention may be used for treating liver carcinoma or colon carcinoma.

The method of the invention uses the combined administration of tyrosine kinase inhibitors and glycolysis inhibitors. It should be appreciated that administration of both compounds includes the administration of each compound alone, in a composition, a combined composition comprising both compounds and any drug combination comprising both compounds. It should be recognized that any of the tyrosine kinase inhibitors disclosed by the invention may be used by the method of the invention.

According to specific embodiments, the tyrosine kinase inhibitor used by the method of the invention may be a compound of formula (XI) or (XII) wherein R1 is isopropyl, R3 is CH3, and R2 is H. As an inhibitor of glycolysis the method of the invention may use at least one of 2-fluoro-deoxyglucos (GI) and 2-deoxyglucose (DG).

In another aspect, the invention further provides a method for the treatment, amelioration, delaying the onset or prophylaxis of a proliferative disorder in a subject being treated with at least one inhibitor of glycolysis. In certain embodiments the method of the invention comprises the step of administering to a subject in need thereof a therapeutically effective amount of at least one inhibitor of tyrosine kinase.

Still further, the invention provides a method for the treatment, amelioration, delaying the onset or prophylaxis of a proliferative disorder in a subject being treated with at least one inhibitor of tyrosine kinase. In some embodiments, the method of the invention comprises the step of administering to a subject in need thereof a therapeutically effective amount of at least one inhibitor of glycolysis.

As used herein, the term “therapeutically effective amount” means an amount of a compound or composition which is administered to a subject in need thereof, necessary to effect a beneficial change in the severity of a disease or disorder, or prevent such disease, in said subject. This amount should also be within specific pharmacological ranges, to avoid toxic effects by over-dosing. For example, in the present invention, a therapeutically effective amount of at least one tyrosine kinase inhibitor and at least one glycolysis inhibitor, for the treatment of cancer would be the amount of these compounds administered to a subject which would induce a beneficial change in the subject, alleviating, ameliorating, or preventing the recurrence of said cancer, without causing detrimental side-effects, or causing only mild side-effects. It is understood that the therapeutically effective amount is not an absolute term and depends on subjective circumstances, such as the subject's age, health, weight, and various other statistics, as described in the and specifically determined by the attendant physician or other person skilled in the art after an evaluation of the subject's conditions and requirements.

It should be further noted that for the method of treatment and prevention provided in the present invention, said therapeutic effective amount, or dosage, is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the at least one tyrosine kinase inhibitor and at least one glycolysis inhibitor used by the invention or any composition of the invention in bodily fluids or tissues. Following successful treatment, it may be desirable to have the patient undergo maintenance therapy to prevent the recurrence of the disease state, wherein the combined composition of the invention is administered in maintenance doses, once, twice, or more daily. Typically, the compositions of the invention (or the combined compositions) may be administered twice a day.

More specifically, the drug combination, combined compositions unit dosage forms used by the methods of the invention containing the at least one tyrosine kinase inhibitor and at least one glycolysis inhibitor, mixture or cocktail thereof, can be administered by the methods of the invention for prophylactic and/or therapeutic treatments. In therapeutic application, compositions are administered to a patient already affected by a proliferative disorder (e.g., colorectal carcinoma and hepato-carcinoma) in an amount sufficient to cure or at least partially arrest the condition and its complications. An amount adequate to accomplish this is defined as a “therapeutically effective dose.” A therapeutically effective dose may be determined, for example, by dose-range finding clinical studies in human patients or, by another example, through determining effective dose in laboratory small or large animals and using one or more or does-conversion methods acceptable in the pharmacological art. As can be appreciated the effective dose may depend on a variety of factors including, but not limited to, nature of disease, disease state, age of patient, sex, gender, concomitant medications, the exact nature of the other drug in which the drug is given in combination which (i.e. the nature of the kinase inhibitor given in combination with a specific glycolysis inhibitor, or vice versa).

For example, amounts effective for this use may range from about 0.1-100 mg/Kg of body weight of the tyrosine kinase inhibitors and about 0.1-100 mg/Kg of body weight the glycolysis inhibitors used according to the invention. Specifically, in embodiments where the compound 0260 is used as the tyrosine kinase inhibitor, the effective amount may range between about 0.1, 0.2, 0.5, 1, and 2 or about 5 mg/Kg body weight to about 10, 20, 50, 90 or about 99 mg/Kg.

In certain embodiments, where a combined treatment uses 0260 in combination with GI as an inhibitor of glycolysis, the effective amount of “compound 0260” may range from about 0.1, 0.2, 0.3, 0.4 or 0.5 to about 1, 2, 3, 4, 5, 6, 7, 8, 9 or 10 mg/Kg. In other embodiments, where the combined treatment includes DG, the effective amount used for compound 0260 may range between about 0.1 to about 50 mg/Kg, about 0.9 to about 30 mg/Kg, about 1 to about 20 mg/Kg, specifically, about 1, 2, 3, 4, 5, 6, 7, 8, 9 or 10 mg/Kg. and more specifically, about 4 mg/Kg. In yet other embodiments, the effective amount of compound 0260 when combined with DG, may be around 50 mg/kg.

In some embodiments, an effective amount of GI may range between about 0.1 to about 20 mg/Kg, about 0.2 to about 8 mg/Kg, about 0.3 to about 6 mg/Kg, about 0.4 to about 4 mg/Kg, about 0.5 to about 2 mg/Kg, about 0.6 to about 1.5 mg/Kg, and specifically, about 1, 1.1, 1.2, 1.3, 1.4, 1.5, 1.6, 1.7, 1.8, 1.9, 2, and more specifically, 1.2 mg/Kg. In other embodiments, the effective amount of GI may be around 15 mg/kg. In other embodiments, an effective amount of DG may range between about 0.1 to about 500 mg/Kg, about 0.2 to about 40 mg/Kg, about 0.3 to about 30 mg/Kg, about 0.4 to about 20 mg/Kg, about 0.5 to about 18 mg/Kg and specifically, about 1, 2, 3, 4, 5, 6, 7, 8, 9, 10, 11, 12, 13, 14, 15, 16, 17, 18, 19 or 20 mg/Kg. and more specifically, about 16 mg/Kg. In other embodiments, the amount of GD may be about 200 mg/kg.

Single or multiple administrations on a daily, biweekly, weekly, every few weeks or monthly schedule can be carried out with dose levels and pattern being selected by the treating physician.

The term “prophylaxis” refers to prevention or reduction the risk of occurrence of the biological or medical event that is sought to be prevented in a tissue, a system, animal or human by a researcher, veterinarian, medical doctor or other clinician, and the term “prophylactically effective amount” is intended to mean that amount of a pharmaceutical composition that will achieve this goal.

The term “prophylactically effective amount” is intended to mean that amount of a pharmaceutical combined composition that will prevent or reduce the risk of occurrence or recurrence of the biological or medical event that is sought to be prevented in a tissue, a system, animal or human by a researcher, veterinarian, medical doctor or other clinician.

In prophylactic applications, drug combinations or compositions containing the tyrosine kinase inhibitor, “compound 0260”, and the glycolysis inhibitors GI and DG, are administered to a patient who is at risk of developing the disease state to enhance the patient's resistance. Such an amount is defined to be a “prophylactically effective dose”. In this use, the precise amounts again depend upon the patient's state of health and general condition, but generally range from 0.1-100 mg/Kg of body weight of the tyrosine kinase inhibitors and about 0.1-100 mg/Kg of body weight the glycolysis inhibitors used by the invention. Specifically, in embodiments where the compound 0260 is used as the tyrosine kinase inhibitor, the effective dosage may range from about 0.4 to about 4 mg/Kg of compound 0260. In other embodiments, the amount of compound 0260 suitable for use may range between about 5 to about 50 mg/kg. GI may range between about 0.1 to about 20 mg/Kg, specifically, about 1.2 mg/Kg. In other embodiments, the amount of GI may be about 15 mg/kg. DG may range between about 0.1 to about 500 mg/Kg, specifically, about 16 mg/Kg. In other specific embodiments, the suitable amount for DG may be about 200 mg/kg.

Single or multiple administrations of the compositions are administered depending on the dosage and frequency as required and tolerated by the patient. In any event, the composition should provide a sufficient quantity of the drug combinations of this invention to effectively treat the patient. Specifically in certain embodiments, double administration is desired, where the dosage mentioned above, is administered at least twice. However, in most cases the dosage is administered periodically until either a therapeutic is achieved and to maintain the therapeutic effect or until side effects warrant discontinuation of therapy. Generally, the dose is sufficient to treat or ameliorate symptoms or signs of disease without producing unacceptable toxicity to the patient.

As indicated above, administration of the active compounds by the method of the invention may utilize the parenteral route (specifically, intraperitoneal administration). However, the method of the invention may be adapted for administration by any other appropriate route, for example by the oral (including buccal or sublingual), rectal, nasal, topical (including buccal, sublingual or transdermal) or vaginal route. Such formulations may be prepared by any method known in the art of pharmacy, for example by bringing into association the active ingredient with the carrier(s) or excipient(s).

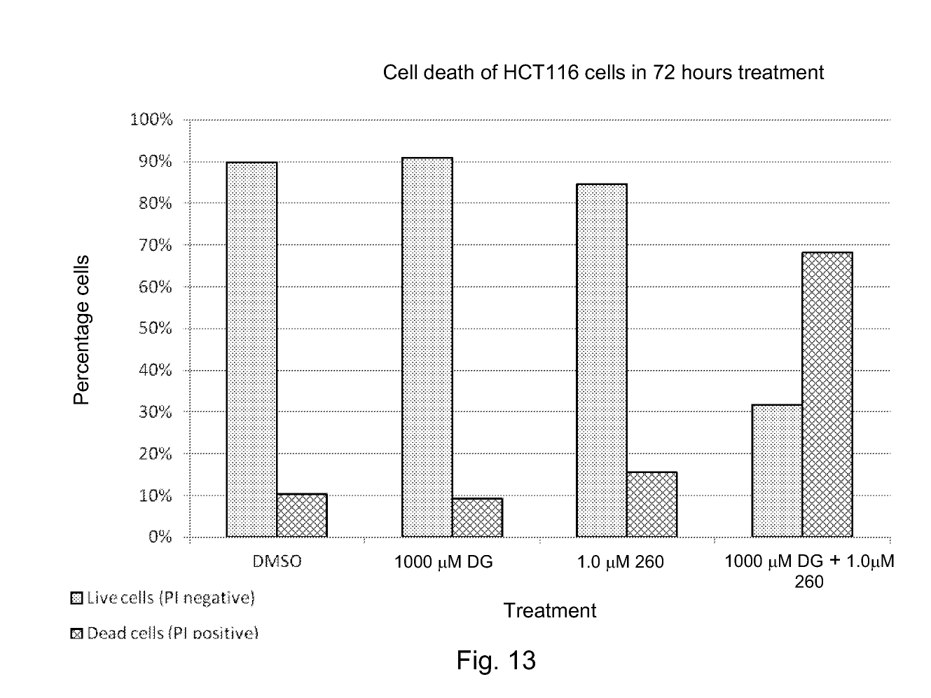

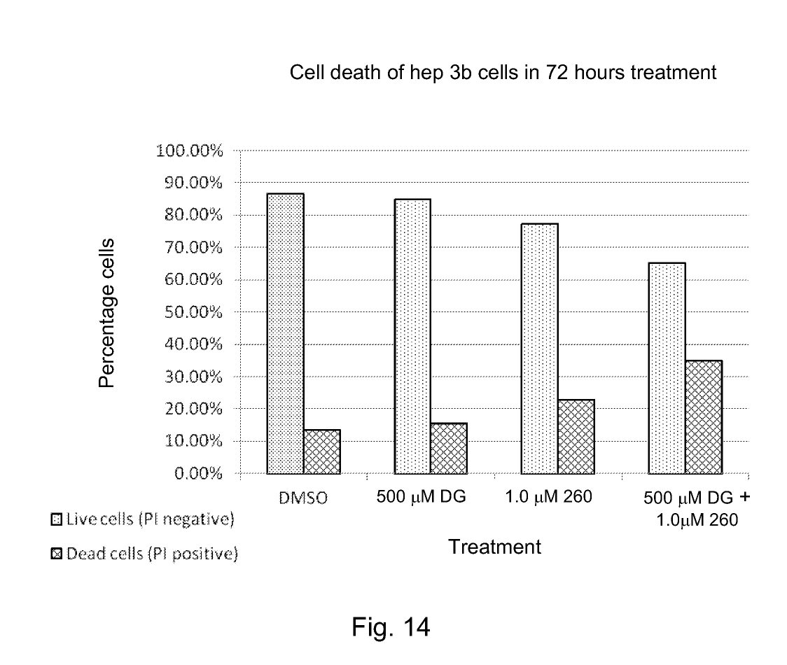

As exemplified in FIGS. 13 and 14, the combined administration of both, the tyrosine kinase inhibitor, compound 0260, and the glycolysis inhibitors GI and DG, clearly led to a synergistic effect on cancerous cell death, most likely, by inducing apoptosis of cancer cells. However, it should be appreciated that although the invention specifically refers to synergistic combinations, the invention also encompasses combinations having an additive effect.

The present invention therefore particularly relates to additive and synergistic combinations of at least one tyrosine kinase inhibitor and at least one glycolysis inhibitor.

Additive and synergistic combinations are useful in treating subjects suffering from a proliferative disorder, for example, Colon cancer or hepato-carcinoma. The synergistic and additive drug combinations or compositions of the invention may also be used for the treatment of subjects presenting with symptoms or signs of such disorders.

By synergic combination is meant that the effect of both tyrosine kinase inhibitor and at least one glycolysis inhibitors is greater than the sum of the therapeutic effects of administration of any of these compounds separately, as a sole treatment.

The combined compounds of the present invention may be administered in the form of a pharmaceutical composition comprising both compounds of this invention together with a pharmaceutically acceptable carrier or diluent. However, according to some embodiments, both compounds may be administered separately. Thus, the compounds used by this invention can be administered either individually in a kit or in a drug combination or alternatively, together in any conventional dosage form.

More particularly, since the present invention relates to the treatment of diseases and conditions with a combination of active ingredients which may be administered separately, the invention also relates as a further aspect, to combining separate pharmaceutical compositions in kit form. The kit includes at least two separate pharmaceutical compositions: (a) at least one inhibitor of tyrosine kinase in a first unit dosage form; and (b) at least one inhibitor of glycolysis in a second unit dosage form.

In some embodiments, the kit of the invention may further comprise a container means for containing said first and second dosage forms.

More specifically, the kit may include container means for containing both separate compositions, such as a divided bottle or a divided foil packet. However, the separate compositions may also be contained within a single, undivided container. Typically the kit includes directions for the administration of the separate components. The kit form is particularly advantageous when the separate components are preferably administered in different dosage forms (e.g., parenteral and oral), are administered at different dosage intervals, or when titration of the individual components of the combination is desired by the prescribing physician.

It should be appreciated that both components of the kit, the at least one tyrosine kinase inhibitor used by the invention, optionally, in the in the first dosage form and the at least one glycolysis inhibitor in the second dosage form may be administered simultaneously.

Alternatively, said first compound or dosage form and said second compound or dosage form may be administered sequentially in either order.

The dosage forms provided in the kits of the present invention can be unit dosage forms wherein the dosage form is intended to deliver one therapeutic dose per administration, e.g., one tablet is equal to one dose. Such dosage forms can be prepared by methods of pharmacy well known to those skilled in the art.

Unit dosage formulations are those containing a daily, biweekly, weekly, every few weeks or monthly dose or sub-dose, as herein above recited, or an appropriate fraction thereof, of an active ingredient. It should be indicated that there are also instances, in which a large bolus dose is given initially and then the dose is reduced for continued treatment.

Preferred unit dosage formulations are those containing a daily dose or sub-dose, as herein above recited, or an appropriate fraction thereof, of an active ingredient.

Achieving a therapeutic effect is meant for example, where the kit is intended for the treatment of a specific disorder, the therapeutic effect may be for example slowing the progression of the treated condition.

Thus, according to certain embodiments, the kit of the invention may be used in the treatment, amelioration, delaying the onset or prophylaxis of a proliferative disorder.

In more specific embodiments, the tyrosine kinase inhibitor comprised within the kit of the invention may be an inhibitor that inhibits the activity of at least one of Fer and FerT. It should be noted that the kit of the invention may use any of the inhibitors disclosed by the invention. In certain embodiment, the kit of the invention may comprise as an inhibitor of Fer and FerT, the compound of formula (XI) or (XII) wherein R1 is isopropyl, R3 is CH3, and R2 is H, and as an inhibitor of glycolysis the kit of the invention may comprise at least one of 2-fluoro-deoxyglucos (GI) and 2-deoxyglucose (DG).

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Materials and Methods

### Reagents

2-fluoro-deoxyglucose (GI)-inhibitors of glycolysis, purchased from SIGMA F5006-100MG.

2-deoxyglucose (DG)-inhibitors of glycolysis, purchased from SIGMA D6134-1G.

“compound 0260”—is the tartarate salt of 6-(4-isopropyl-phenyl)-2-{4-[(4-methyl-piperazin-1-yl)methyl]piperidin-1-yl}imidazo[2,1-b][1,3,4]thiadiazole. This Fer inhibitor is also referred to as compound “522-0251” in WO 2010/097798.

### Cells and Cell Lines

Various combinations of inhibitors of glycolysis and tyrosine kinase inhibitors were presented to the following cancer cell lines: Hep3B liver cancer cells, SW620 colon cancer cells, and HCT116 colon cancer cells. Human foreskin cells FS11 served as a non-cancerous control cell population.

### Experimental Procedures

### XXT Assay for Cell Viability

Cells were seeded in 96 well plates, and the various test substances, as indicated below, were added. After 72 hours the medium was removed and 50 μl of the reagent XXT (Dimethylthiazol-2-yl]-2,5-diphenyl tetrazolium) was added, and the cells were incubated for 2 hours at 37° C. The method relies on the ability of cells to take up the soluble tetrazolium salt (which is yellow). In living cells, the tetrazolium ring is broken down into by dihydrogenase in the mitochondria and is reduced to formazan, which is blue. The blue dye was quantified in an ELISA reader at 540 nm.

### Xenografting in Mice

Tumors originating from cancer cells were induced in ATHYMIC/NU nude mice, which lack T cells, by subcutaneous injection. Seven days after the injection, and after the appearance of tumors, the mice were treated with various test substances by intraperitoneal injection. In control mice, only the solvent was injected intraperitoneally.

### Cell Cycle Analysis

Florescence assisted cell sorting (FACS) was performed as described in Pasder O, et al. [Pasder O, et al. Oncogene 2006; 25(30):4194-206]. Cells were rinsed twice with cold PBS, and then harvested by treatment with trypsin. Suspended cells were washed twice in 10 ml cold PBS and centrifuged at 500 g for 5 min. In cell cycle analyses, the pellet was resuspended in 200 μl cold PBS and cells were stained in the dark with 200 μl propidium iodide (PI) solution (20 mM Tris, pH 8, 1 mM NaCl, 0.1% (v/v) NP-40, 1.4 mg/ml RNase A, 0.05 mg/ml PI) for 30 min at 371C. In experiments to determine the fraction of dead cells in a population of cells, the detergent NP-40 was omitted. The total cellular DNA content was determined using a Becton Dickinson flow cytometer (FACSCalibur). The data were analyzed using Cell Quest Pro software (Becton Dickinson) and ModFit LT software.

### Immunoprecipitation (IP) Analysis

After extraction of cellular proteins, a protein sample of 750-1000 μg was diluted 3fold with TGET buffer (20 mM Tris-HCl (Ph7.5), 1 mM EDTA, 0.1% Triton X100 and 10% Glycerol) with 150 nM NaCl and protease inhibitors and incubated with the indicated antibody at a dilution of 1:100 overnight at 4° C. with shaking. At the end of the incubation, 25 μl Protein A/G suspension was added bound to agarose beads. The mixture was shaken for another 90 min at 4° C. The immune complex precipitate was obtained after centrifugation for 20 min at maximum speed and then washed three times in extraction buffer: twice with buffer TGET NaCl 150 mM and once with TGET NaCl 75 mM. The precipitate was suspended in 2× loading buffer. After boiling for 5 min the proteins were separated by SDS PAGE and transferred to a nitrocellulose membrane and reacted with the indicated antibodies.

Preparation of siRNA

Double-stranded RNAs, 21 nucleotides long, were synthesized by Dharmacon Research (Lafayette, Colo., USA). Custom SMART pool siRNA to target the human Fer mRNA and the human FerT mRNA were purchased from Dharmacon. Selected siRNAs sequences were submitted to a BLAST search against the human genome sequence to ensure specificity of the siRNA. A sequence targeting firefly (Photinus pyralis) luciferase (luc.) gene (Accession no. X65324) was used as a control. Fer and ferT siRNAs are denoted by SEQ ID Nos. 4 and 5, respectively.

siRNA Transfection

For siRNA transfection, 1.5×10⁵ HCT1160 cells were seeded in 6 cm Petri dish one day before transfection. 30 μl siRNA from 20 μM stock solution were mixed with 300 μl OptiMEM (Gibco-Invitrogen) medium, then incubated at room temperature for 5 min. 20 μl Metafectene (Biontex) were mixed with 160 μl OptiMEM in a separate tube and were also incubated at room temperature for 5 min. The two mixtures were then combined, gently mixed, and incubated for another 15 min at room temperature, for the formation of lipids-siRNA complexes. Growth medium was removed from the cells, which were then covered with 1290 μl OptiMEM, 200 μl FCS, and 510 μl lipid-siRNA mixture. Final siRNA concentration in the Petri dish was 300 nM. 8 hours later, 1800 μl Optimem and 200 μl FCS were added and cells were harvested 72 h after transfection.

### ROS Levels and Complex I and V Activity

Cellular levels of Reactive oxygen species (ROS) were determined using Oxiselect ROS assay kit (Cell Biolabs, INC.).
Complex I activity and complex V ATP synthesizing activity were determined using specific Microplate assays (MS141 and MS543—MitoSciences, respectively).

### Immunostaining Studies

HCT116 CC cells were fixed and co-immuno-stained with anti-mitochondria mono-clonal antibody (AE-1 Meridian-green) and with an anti-Fer antibody (red). Obtained images were processed using the IMRIS application.

### Example 1

### Mitochondrial Expression and Function of Fer and FerT in Cancer Cells

Cancer cells adopt mitochondrial alterations and metabolic re-programming in order to sustain their unique metabolic needs and produce all molecules and energy required to promote tumor growth. Intriguingly, while the central role of aerobic glycolysis in cancer cells has been well documented, inhibition of the glycolytic pathway did not always lead to an effective therapeutic outcome. Thus, cancer cells might find the way to stimulate their mitochondrial energy generation system under glycolysis restrictive conditions. Accordingly, attempts are being made to identify mitochondrial components that could be linked to the abnormal functioning of this organelle in cancer cells.

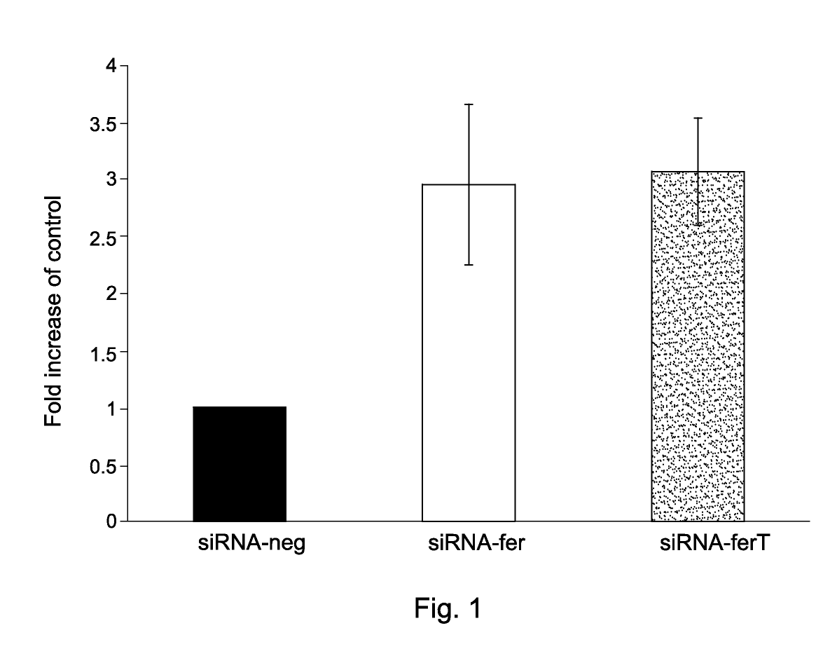

In normal cells, the tyrosine kinase Fer is found in the mitochondria while its short meiotic isoform-FerT is normally expressed only in meiotic cells in the testis. However, while being absent from normal somatic tissues, FerT was surprisingly found by the present invention to accumulate in colon carcinoma (CC) and in hepato-cellular carcinoma (HCC) cells. To further examine the mitochondrial role of Fer and Fer T, Knock down experiment were next performed. As clearly demonstrated in FIG. 1, down-regulation of Fer and FerT induce ROS levels. This effect was accompanied by cell-cycle arrest and apoptotic death in CC cells (not shown).

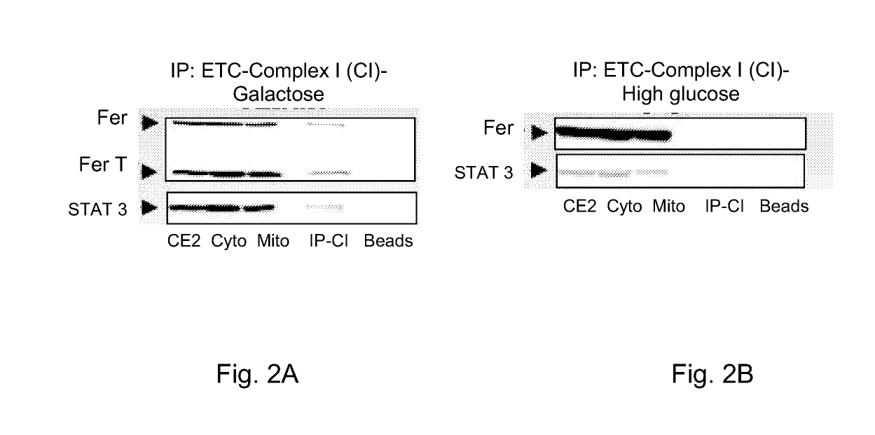

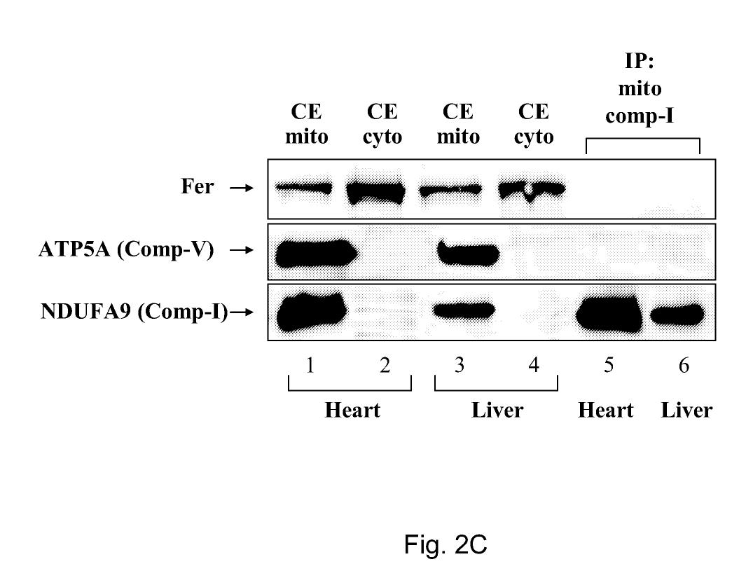

Since one of the major sources of ROS production in cancer cells is the mitochondrion, the inventors next examined whether the restraining effect of Fer and FerT on ROS production reflects their mitochondrial related functions in CC cells. A mentioned above, by applying biochemical and immunocytochemical analyses the inventors found that both Fer and FerT reside in the mitochondria of malignant cells. More specifically, whole cell extracts (CE2) from Sw620 colon carcinoma cells were fractionated to cytoplasmic (cyto) and mitochondrial (Mito) fractions. Proteins were extracted from each fraction and reacted with anti-Fer/FerT antibody. FIG. 2 (A and B), clearly demonstrates the mitochondrial expression of both Fer and FerT, however, only Fer is expressed in normal tissue (liver and heart, shown in FIG. 2C). The Immunostaining presented in FIG. 3, provides further support for the elevated expression of Fer and FerT proteins in mitochondria of malignant HCT116 colon cancer cells.

Co-expression of Fer and FerT in malignant cells indicate that these enzymes may be involved in mitochondrial functions. Therefore, the association of both enzymes with the electron transport chain (ETC) complex I (CI) was next examined by co-immunoprecipitation (co-IP) studies. As shown in FIG. 2A, the association of Fer and FerT with this complex is best observed when cells are grown with galactose without glucose (compare FIGS. 2A and 2B), conditions under which energy generation in cancer cells is shifted toward the mitochondria. The transcription-factor-Signal transducer and activator of transcription 3 (Stat3), served as positive control. It should be noted that no association was observed in normal cells (FIG. 2C).

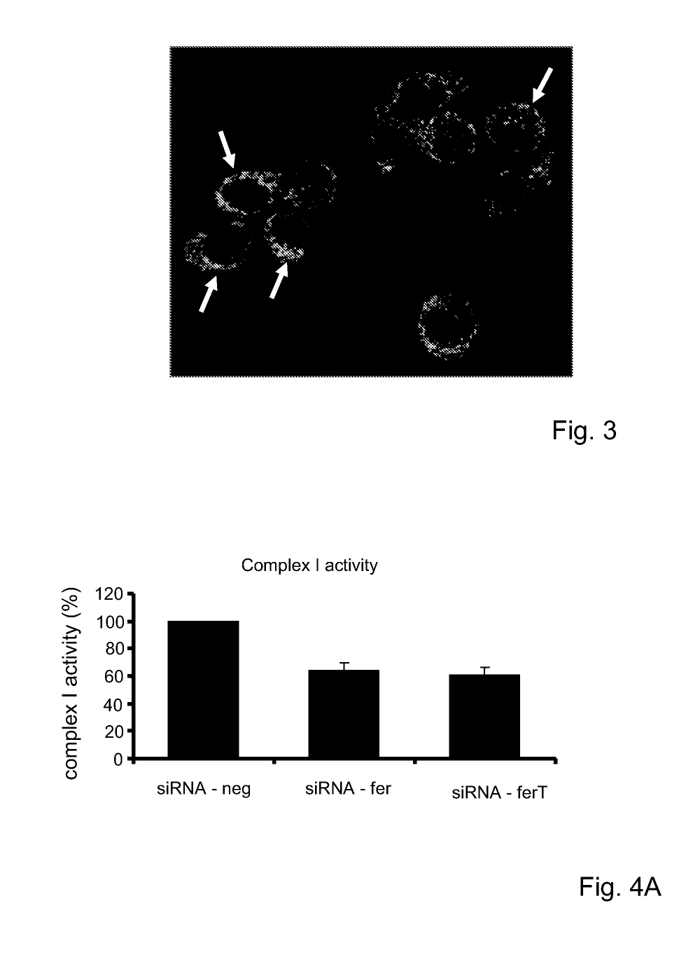

Moreover, knock-down of Fer and FerT under glycolysis restricting conditions clearly decreased the activity of this complex (FIG. 4A). Furthermore, as shown in FIG. 4B, the reduction in complex I activity was accompanied by an impaired production of ATP by the mitochondrial ETC complex V.

These results imply that targeting of Fer and FerT in parallel to glycolysis attenuation could significantly and selectively impair energy production in cancer cells.

### Example 2

### Combined Effect of Fer and Glycolysis Inhibitors on Cancer Cells In Vitro

To translate these findings into a new cancer therapeutic approach the inventors sought to inhibit the energy production in malignant cells by blocking simultaneously their glycolytic pathway and the potential Fer/FerT mediated revival of their mitochondrial energy generation system. This can be achieved by combined subjection of malignant cells to the competitive hexokinase II inhibitor and attenuator of glycolysis, together with an inhibitor of Fer and FerT.

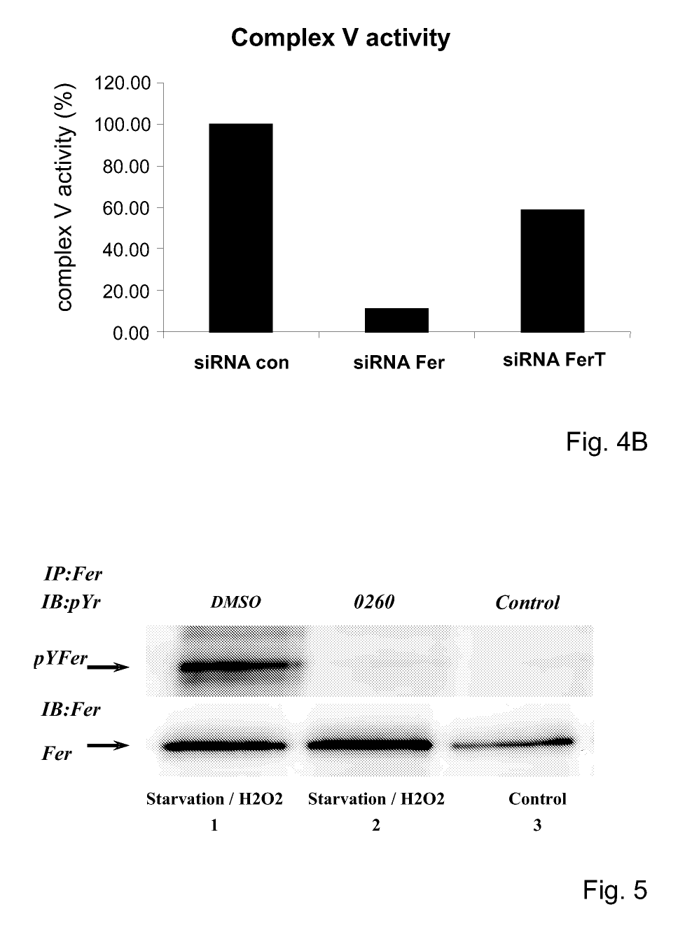

A synthetic inhibitor of Fer and FerT, previously developed by the inventors was used for these combined experiments. As shown by FIG. 5, the 0260 compound exhibits an efficient inhibition of this tyrosine kinase as demonstrated using an in-vivo Fer activity assay. As inhibitors of glycolysis 2-fluoro-deoxyglucose (GI) and 2-deoxyglucose (DG) were used.

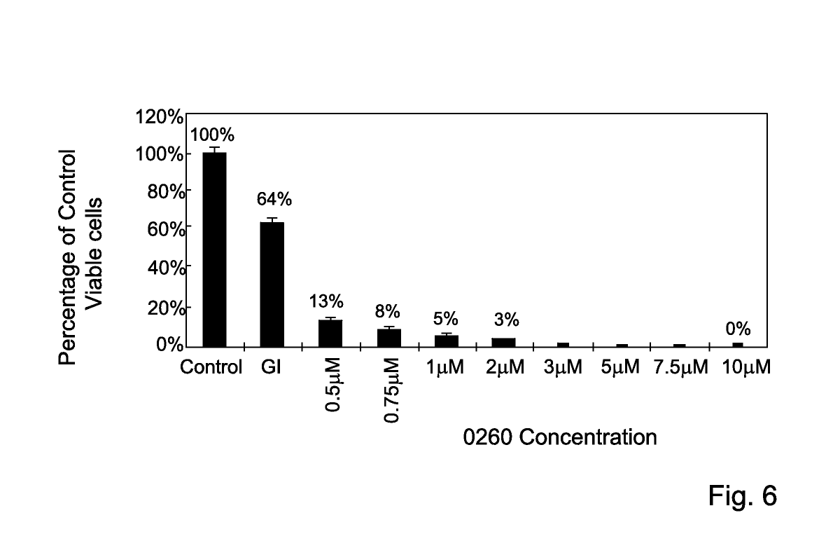

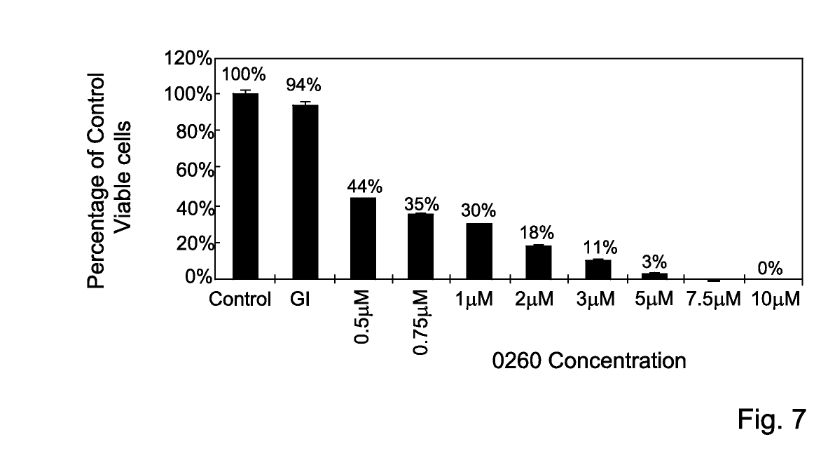

In vitro studies of the combined effect of Fer and glycolysis inhibitors were preformed in Hep3B liver cancer cells treated with 100 μM 2-F-deoxyglucose GI (glycolysis inhibitor) and increasing concentrations of 0260 for 72 h. The percentage of viable cells was determined using XTT assay. As shown in FIG. 6, simultaneous administration of the two inhibitors reduced the rate of proliferation of in Hep3B liver cancer cells. Similar results were observed in SW620 colon cancer cells (FIG. 7).

### Example 3

### In Vivo Combined Effect of Fer and Glycolysis Inhibitors on Tumors

In vivo studies of the combined effect of Fer and glycolysis inhibitors were carried out using the xenograft model of human Hep3B liver cancer cells in immuno-compromised nude mice. Mice were then treated twice a day with IP injection of 2-fluoro-deoxyglucose (GI) (15 mg/kg), and compound 0260 (5 mg/kg) or with the solvent −20% chremophor EL (vehicle). Each group contained 8 animals and the average volume of all tumors in each group is presented. As shown in FIG. 8, simultaneous administration of the two inhibitors significantly attenuated the progression of the human liver cancer xenografts in mice.

FIG. 9 demonstrates similar experiments using a combination of an alternative glycolysis inhibitor 2-deoxyglucose DG (200 mg/kg) with 0260 (50 mg/kg) injected IP twice a day to nude mice with human Hep3B xenografts. These sets of experiments strongly support the notion that therapeutic approach directed at inhibition of glycolysis and Fer activity is highly effective for attenuation of liver cancer in vivo. Similar experiments are now carried out using the xenograft model of the HCT116 colon cancer cells in nude mice.

### Example 4

### Specific Effect Fer and Glycolysis Inhibitors Combination on Cancer Cells

The inventors previously showed that down-regulation of Fer induce phosphoprotein phosphatase 1 (PP1) activation and cell-cycle arrest in malignant cells (WO 2007/107991). To study the effect of various combinations of Fer and glycolysis inhibitors on cell cycle progression, various concentrations of 0260 and DG alone and in combination were tested in Hep3B liver cancer cells and on human foreskin cells (FS11) as a non-cancer controls.

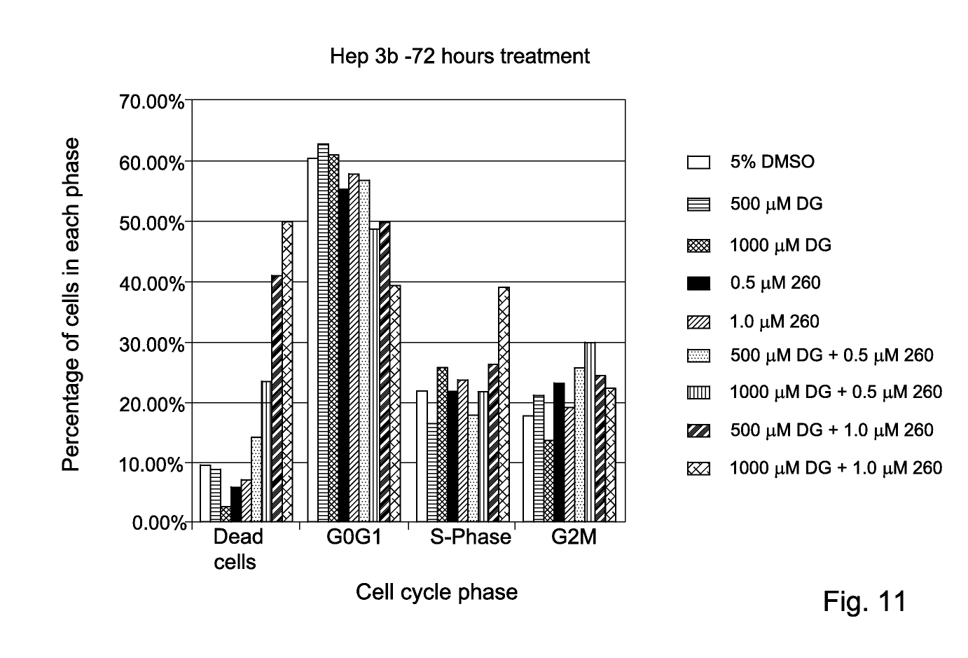

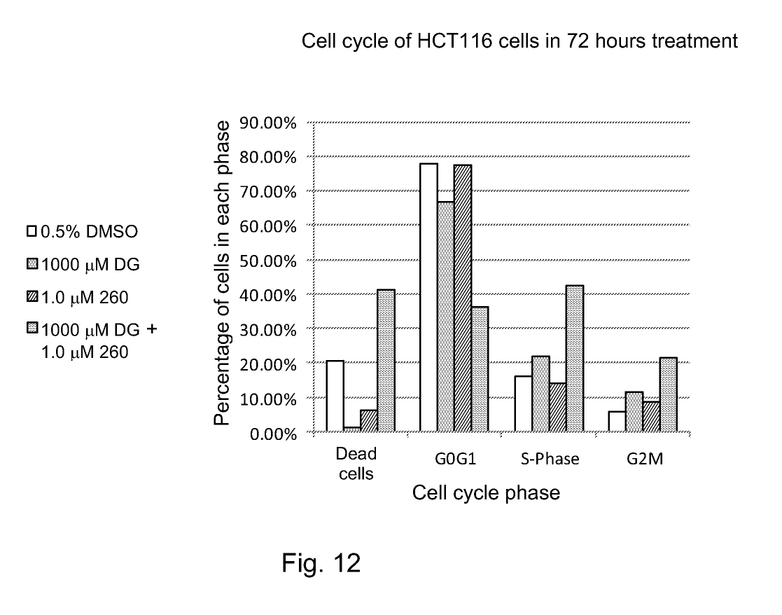

FIG. 10. shows that even at the highest concentrations (1000 μM DG and 1 μM compound 0260) alone, or in different combinations had only minor effects on the dead cells fraction in normal FS11 cells. In contrast, combination of 0260 and DG significantly increased the dead cells fraction of malignant cells, as clearly shown for Hep3B liver cancer cells in FIG. 11. Similar effect of the 0260 and DG combination was shown in FIG. 12 for HCT116 colon cancer cells. These experiments suggest that while inhibition of Fer and glycolysis is probably nontoxic to normal cells, it is highly effective in cancer cells of various types.

### Example 5

### Synergistic Effect of Fer and Glycolysis Inhibitors Combination

While looking at the additive effect of 0260 and DG combination on the fraction of dead cells in a population of malignant Hep3B or HCT116 cells in FIGS. 13 and 14, it became evident that when presented alone each compound had only minor effect on the dead cells fraction. However in combination, the two compounds induced a significant increase in the number of dead cells, in HCT116 the dead cells fraction exceeded the live cells fraction. These experiments strongly support the notion that inhibition of glycolysis and Fer activity has a synergistic effect on driving cancer cells to apoptosis.

1. A drug combination comprising:
(a) an effective amount of at least one inhibitor of tyrosine kinase; and
(b) an effective amount of at least one inhibitor of glycolysis.

2. The drug combination according to claim 1, wherein said inhibitor of tyrosine kinase is an inhibitor of at least one of Fer and FerT.

3. The drug combination according to claim 2, wherein said inhibitor of at least one of Fer and FerT is a compound of formula (I) or pharmaceutically acceptable salts thereof: 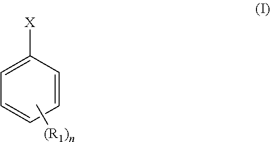
wherein X is selected from the following: 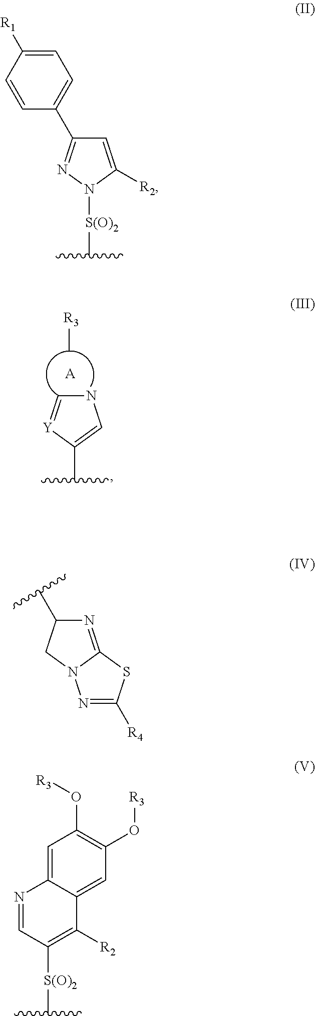
wherein R₁ is independently selected from H, F, Cl, Br, I or a C₁₋₅ linear or branched alkyl; n is 1, 2, 3, 4 or 5; R₂ is N(R)₂, R being independently hydrogen or linear or branched C₁₋₅alkyl group; R₃ is a linear or branched C₁₋₅alkyl group; R₄ is a group of formula 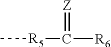
R₅ being a 5- or 6-membered aromatic or non-aromatic ring optionally having one, two or three heteroatoms selected from O, N or S; R₆ being a 5- or 6-membered aromatic or non-aromatic ring optionally having one, two or three heteroatoms selected from O, N or S optionally having one or two substituents independently selected from halogen and a linear or branched C₁₋₅alkyl group; Z is selected from O or S; Y is C—H or N; and A is a 5- or 6-membered fused aromatic or non aromatic ring optionally being a heterocyclic ring comprising 1 to 3 heteroatoms selected from O, N or S;
C₁₋₅ Linear or branched alkyl means a methyl, ethyl, propyl, isopropyl, n-butyl, isobutyl, sec.-butyl, tert.-butyl, n-pentane, iso-pentane, sec.-pentane or tert.-pentane that may optionally be partially substituted by halogen selected from F, Cl, Br, or I.

4. The drug combination according to claim 3, wherein the tyrosine kinase inhibitor is a compound having the chemical formula C₂₂H₂₈N₆OS (X):

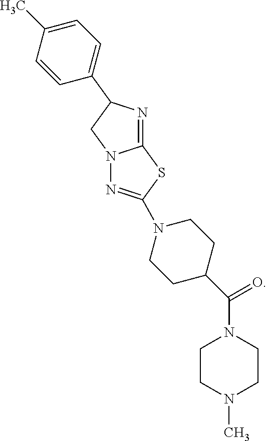 [SMILES: Cc1ccc(C2CN3N=C(N4CCC(C(=O)N5CCN(C)CC5)CC4)SC3=N2)cc1]

5. The drug combination according to claim 4, wherein said tyrosine kinase inhibitor is a compound of formulae (XI) or (XII) or any pharmaceutically acceptable salts thereof: 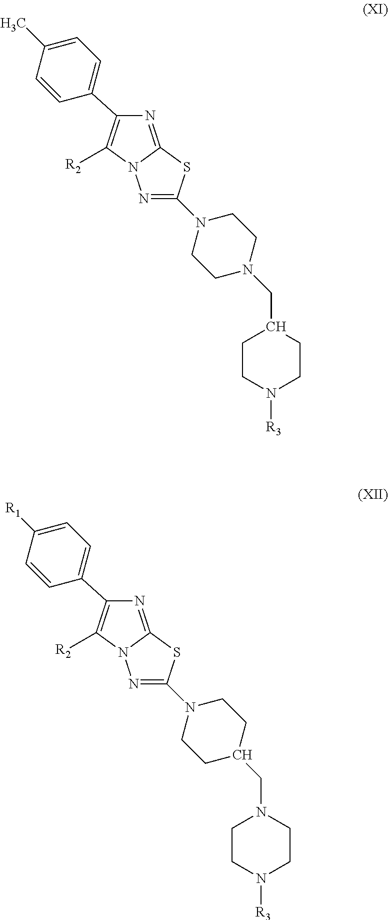
wherein R₁, R₂ and R₃ are independently selected from hydrogen, halogen, C₁₋₆alkyl, C₂₋₆alkenyl, N—C₁₋₆alkyl, N—C₂₋₆alkenyl the C₁₋₆alkyl and C₂₋₆alkenyl being straight or branched.

6. The drug combination according to claim 5, wherein said tyrosine kinase inhibitor is a compound of formula (XI) or (XII) wherein R1 is isopropyl, R3 is CH3, and R2 is H.

7. The drug combination according to claim 1, wherein said inhibitor of glycolysis is at least one of 2-fluoro-deoxyglucos (GI) and 2-deoxyglucose (DG).

8. The drug combination according to claim 1, for the treatment, amelioration, delaying the onset or prophylaxis of a proliferative disorder.

9. The drug combination according to claim 8, wherein proliferative disorder is any one of liver carcinoma and colon carcinoma.

10. A pharmaceutical composition comprising an effective amount of at least one inhibitor of tyrosine kinase, an effective amount of at least one inhibitor of glycolysis and a physiologically acceptable carrier.

11. The pharmaceutical composition according to claim 10, wherein said tyrosine kinase inhibitor is a compound of formula (XI) or (XII) wherein R1 is isopropyl, R3 is CH3, and R2 is H, and wherein said inhibitor of glycolysis is at least one of 2-fluoro-deoxyglucos (GI) and 2-deoxyglucose (DG).

12. The composition according to claim 11, for the treatment, amelioration, delaying the onset or prophylaxis of a proliferative disorder.

13. The composition according to claim 12, wherein said proliferative disorder is any one of liver carcinoma and colon carcinoma.

14. A method for the treatment, amelioration, delaying the onset or prophylaxis of a proliferative disorder comprising administering to a subject in need thereof a therapeutically effective amount of at least one inhibitor of tyrosine kinase and an effective amount of at least one inhibitor of glycolysis, any combination thereof or any composition comprising the same.

15. The method according to claim 14, wherein proliferative disorder is any one of liver carcinoma and colon carcinoma.

16. The method according to claim 14, wherein said tyrosine kinase inhibitor is a compound of formula (XI) or (XII) wherein R1 is isopropyl, R3 is CH3, and R2 is H, and wherein said inhibitor of glycolysis is at least one of 2-fluoro-deoxyglucos (GI) and 2-deoxyglucose (DG).

17. A method for the treatment, amelioration, delaying the onset or prophylaxis of a proliferative disorder in a subject being treated with at least one inhibitor of glycolysis comprising the step of administering to a subject in need thereof a therapeutically effective amount of at least one inhibitor of tyrosine kinase.

18. A method for the treatment, amelioration, delaying the onset or prophylaxis of a proliferative disorder in a subject being treated with at least one inhibitor of tyrosine kinase comprising the step of administering to a subject in need thereof a therapeutically effective amount of at least one inhibitor of glycolysis.

19. A kit comprising:
(a) at least one inhibitor of tyrosine kinase in a first unit dosage form; and
(b) at least one inhibitor of glycolysis in a second unit dosage form.

20. The kit according to claim 19, comprising a container means for containing said first and second dosage forms.

21. The kit according to claim 19, for the treatment, amelioration, delaying the onset or prophylaxis of a proliferative disorder.

22. The kit according to claim 21, wherein said tyrosine kinase inhibitor is a compound of formula (XI) or (XII) wherein R1 is isopropyl, R3 is CH3, and R2 is H, and wherein said inhibitor of glycolysis is at least one of 2-fluoro-deoxyglucos (GI) and 2-deoxyglucose (DG).
